(19) United States
(12) Patent Application Publication
(10) Pub. No.: US 2009/0048282 A1
(43) Pub. Date: Feb. 19, 2009

(54) PYRIMIDINE SULFONAMIDE ANALOGS AND THEIR USE AS AGONISTS OF THE WNT-BETA-CATENIN CELLULAR MESSAGING SYSTEM

(75) Inventors: Diane Barbara Hauze, Wayne, PA (US); Matthew Gregory Bursavich, Salt Lake City, UT (US); Luciana de Araujo Felix, Broomall, PA (US); Adam Matthew Gilbert, Congers, NY (US); Jeffrey Claude Pelletier, Lafayette Hill, PA (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/191,388

(22) Filed: Aug. 14, 2008

Related U.S. Application Data
(60) Provisional application No. 60/964,735, filed on Aug. 14, 2007.

(51) Int. Cl. A61K 31/506; A61P 35/00; C07D 417/02

(52) U.S. Cl. 514/275; 544/330; 544/331

(57) ABSTRACT

The present invention relates to pyrimidine sulfonamide analogs, methods of making pyrimidine sulfonamide analogs, compositions comprising a pyrimidine sulfonamide analog, and methods for treating canonical Wnt-β-catenin cellular messaging system-related disorders comprising administering to a subject in need thereof an effective amount of a pyrimidine sulfonamide analog.

### CROSS-REFERENCE TO RELATED APPLICATIONS

This application claims priority benefit of U.S. provisional patent application No. 60/964,735, filed Aug. 14, 2007, the entire disclosure of which is hereby incorporated by reference.

### FIELD OF THE INVENTION

The invention relates to heteroaryl/aryl pyrimidine analogs, compositions comprising a heteroaryl/aryl pyrimidine analog, and methods for treating or preventing disorder involving the canonical Wnt-β-catenin cellular messaging system comprising the administration of an effective amount of a heteroaryl/aryl pyrimidine analog.

### BACKGROUND OF THE INVENTION

The Wnt-β-catenin cellular messaging system is essential in many biological processes. It regulates the fate of as-yet undeveloped cells in embryo form. The signals in the messaging system also direct the development of stem cells in adult organisms (e.g. skin cell, bone cell, liver cell, etc.). At the cellular level, the canonical Wnt-β-catenin cellular messaging system regulates morphology, proliferation, motility and cell fate. The Wnt-β-catenin messaging system has a central role in tumorigenesis and inappropriate activation of this system is observed in several human cancers.

Wnt-β-catenin was first described in humans as a protein, which interacts with the cytoplasmic domain of E-cadherin and with α-catenin, anchoring the cadherin complex to the actin cytoskeleton. Then, an additional role for mammalian β-catenin was discovered; namely, as the key mediator of Wnt-β-catenin messaging.

Chronic activation of the Wnt-β-catenin cellular messaging system has been implicated in the development of a variety of human malignancies, including colorectal carcinomas, hepatocellular carcinomas (HCCs), melanomas, and uterine and ovarian carcinomas.

The Wnt-β-catenin cellular messaging system also plays a role in degenerative diseases such as Alzheimer's disease (AD) and bone disorders.

AD is the most common age-related neurodegenerative disorder. A massive accumulation of beta-amyloid (Abeta) peptide aggregates is likely the pivotal event in AD. Abeta-induced toxicity is accompanied by a varied combination of events including oxidative stress. The Wnt-β-catenin pathway has multiple actions in the cascade of events triggered by Abeta, and drugs with Wnt-β-catenin activity can be therapeutics for AD treatment.

Various bone disorders are also associated with defects in the Wnt-β-catenin messaging system. Signaling through the Wnt-β-catenin pathway increases bone mass through a number of mechanisms, including renewal of stem cells, stimulation of preosteoblast replication, induction of osteoblastogenesis, and inhibition of osteoblast and osteocyte apoptosis.

As discussed above, agonists of the Wnt-β-catenin messaging system are expected to be medicaments useful against cell proliferation disorders, bone disorders, and Alzheimer's disease. Thus, it would be advantageous to have novel agonists of the Wnt-β-catenin messaging system as potential treatment regimens for Wnt-β-catenin messaging system-related diseases. The instant invention is directed to these and other important ends.

### SUMMARY OF THE INVENTION

In one aspect, the invention provides compounds of the Formula (I):

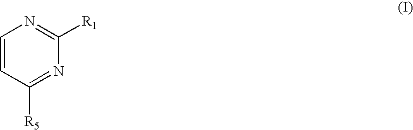

and pharmaceutically acceptable salts thereof,
wherein

R₅ is aryl, heteroaryl or C₄-C₈ cycloalkenyl both optionally substituted with 1-7 R₄ groups; and

R₁ is

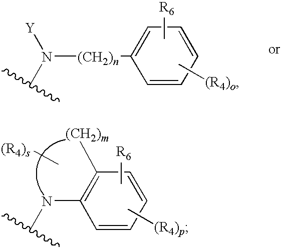

wherein
Y is H, C₁₋₆ alkyl, aryl, or arylalkyl;
R₆ is —SO₂NR₂R₃ or

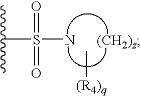

R₂ and R₃ are each independently H, C₁₋₆ alkyl, C₂₋₆ alkenyl, C₂₋₆ alkynyl, C₃₋₆cycloalkyl, C₃₋₆ cycloalkyl-C₁₋₃ alkyl, aryl, arylalkyl, heteroaryl, or heteroarylalkyl, whereas all except H can be optionally substituted with 1-5 R₄ groups;

each R₄ is independently H, halogen, CN, OH, aryl, arylalkyl, heteroaryl, heteroarylalkyl, C₁₋₆ alkyl, C₂₋₆ alkenyl optionally substituted with di(C₁-C₆)alkylaminocarbonyl or with di(C₁-C₆)alkylamino-(C₁-C₆)alkyloxycarbonyl, C₂₋₆ alkynyl optionally substituted with heteroaryl, C₁₋₃ fluorinatedalkyl, C₃₋₆ cycloalkyl, C₃₋₆ cycloalkyl-C₁₋₃ alkyl, NO₂, NH₂, NHC₁₋₆ alkyl, N(C₁₋₆ alkyl)₂, NHC₃₋₆ cycloalkyl, N(C₃₋₆ cycloalkyl)₂, NHC(O)C₁₋₆ alkyl, NHC(O)C₃₋₆ cycloalkyl, NHC(O)NHC₁₋₆ alkyl, NHC(O)NHC₃₋₆ cycloalkyl, SO₂NH₂, SO₂NHC₁₋₆ alkyl, SO₂NHC₃₋₆ cycloalkyl, SO₂N(C₁₋₆ alkyl)₂, SO₂N(aryl-C₁₋₆ alkyl)₂, SO₂N(C₃₋₆ cycloalkyl)₂, NHSO₂C₁₋₆ alkyl, NHSO₂C₃₋₆ cycloalkyl, CO₂H. CO₂C₁₋₆ alkyl, CO₂C₃₋₆ cycloalkyl, CONHC₁₋₆ alkyl, CONHC₃₋₆ cycloalkyl, CON(C₁₋₆ alkyl)₂, CON(C₃₋₆ cycloalkyl)₂OH, OC₁₋₃ alkyl, C₁₋₃ fluorinatedalkyl, OC₃₋₆ cycloalkyl, OC₃₋₆ cycloalkyl-C₁₋₃ alkyl, C₁₋₃alkyloxy-C₁₋₃ alkyl, SH, SOₓC₁₋₃ alkyl, C₃₋₆ cycloalkyl, or SOₓC₃₋₆ cycloalkyl-C₁₋₃ alkyl;
or if R₄ and R₆ are bonded to phenyl ring carbons that are adjacent to each other, then R₄ and R₆ taken together with the two phenyl ring carbons form a heteroaromatic ring containing an —SO₂—NH—, an —SO₂—N(C₁-C₆ alkyl)-, or an —SO₂—N(aryl)-;
n is 0 or 1;
m is 2 or 3;
o is 0, 1, 2, 3, or 4;
p is 0, 1, 2, 3, 4, 5, or 6;
q is 0, 1, 2, 3, 4, 5, 6, 7, 8, 9, 10, 11, or 12;
s is 0, 1, 2, 3, 4, 5, or 6;
x is 0, 1, or 2; and
z is 3, 4, 5, or 6.

In another aspect, the invention provides compounds of the formula (II):

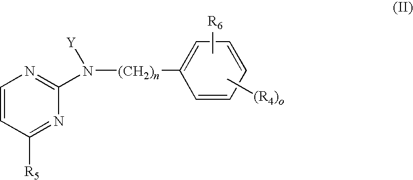

and pharmaceutically acceptable salts thereof,
wherein

R₄, R₅, R₆, n, o, and Y are as defined above.

In another aspect, the invention provides compounds of the formula (III):

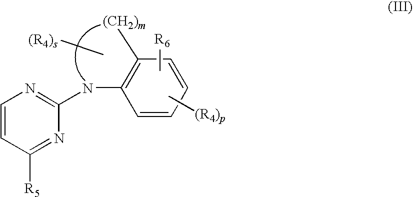

and pharmaceutically acceptable salts thereof,
wherein

R₄, R₅, R₆, m, p, and s are as defined above.

In another aspect, the invention provides methods of synthesizing compounds of Formula (II) comprising:

reacting a compound of the Formula (IV):

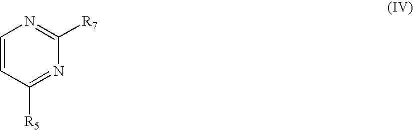

wherein

R₅ is as defined above; and

R₇ is halogen;

with a compound of Formula (V):

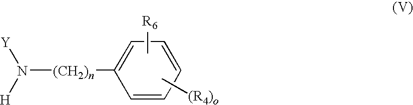

wherein
n, Y, R₄, o, and R₆ are as defined above;
under conditions effective to substitute R₇ with the compound of Formula (V) thereby providing a compound having the Formula (II):

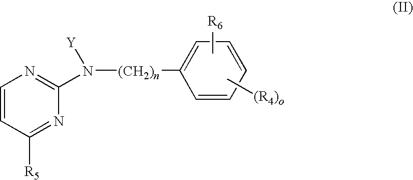

and pharmaceutically acceptable salts thereof,
wherein

R₄, R₅, R₆, n, o, and Y are as defined above.

In another aspect, the invention provides methods of synthesizing compounds of Formula (III) comprising:

reacting a compound of the Formula (IV):

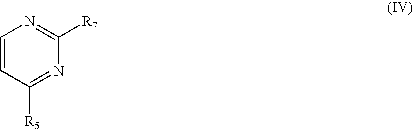

wherein

R₅ and R₇ are as defined above;

with a compound of Formula (VI):

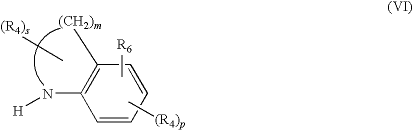

wherein

R₄, R₆, m, p, and s are as defined above.

under conditions effective to substitute R₇ with the compound of Formula (VI) thereby providing a compound having the Formula (III):

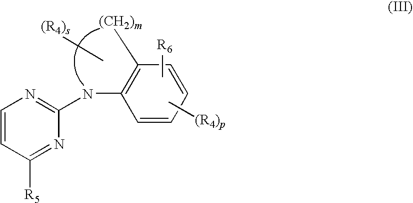

and pharmaceutically acceptable salts thereof,
wherein

R₄, R₅, R₆, m, p, s are as defined above.

In other aspects, the invention provides pharmaceutical compositions comprising compounds or pharmaceutically acceptable salts, hydrates, or solvates of compounds of Formula (I), Formula (II), and Formula (III) and a pharmaceutically acceptable carrier.

In other aspects, the compounds or pharmaceutically acceptable salts thereof of the compounds of Formula (I), Formula (II), and Formula (III) are useful as canonical Wnt-β-catenin cellular messaging system agonists.

In other aspects, the invention provides methods for treating a canonical Wnt-β-catenin cellular messaging system related disorder, comprising administering to a mammal in need thereof a compound or a pharmaceutically acceptable salt of a compound of Formula (I), Formula (II), and Formula (III) in an amount effective to treat a canonical Wnt-β-catenin cellular messaging system related disorder.

### DETAILED DESCRIPTION OF THE INVENTION

The following definitions are used in connection with the pyrimidine sulfonamide analogs of the present invention:

“Alkyl” refers to a hydrocarbon chain that may be a straight chain or branched chain, containing the indicated number of carbon atoms. For example, C₁-C₆ indicates that the group may have from 1 to 6 (inclusive) carbon atoms in it.

“Aryl” refers to cyclic aromatic carbon ring systems made from 6 to 18 carbons. Examples of an aryl group include, but are not limited to, phenyl, naphthyl, anthracenyl, tetracenyl, and phenanthrenyl. An aryl group can be unsubstituted or substituted with one or more of the following groups: halogen, CN, OH, aryl, arylalkyl, heteroaryl, heteroarylalkyl, C₁₋₆ alkyl, C₂₋₆ alkenyl, C₂₋₆ alkynyl, C₁₋₃ fluorinatedalkyl, C₃₋₆ cycloalkyl, C₃₋₆cycloalkyl-C₁₋₃alkyl, NO₂, NH₂, NHC₁₋₆ alkyl, N(C₁₋₆ alkyl)₂, NHC₃₋₆ cycloalkyl, N(C₃₋₆ cycloalkyl)₂, NHC(O)C₁₋₆ alkyl, NHC(O)C₃₋₆ cycloalkyl, NHC(O)NHC₁₋₆ alkyl, NHC(O)NHC₃₋₆ cycloalkyl, SO₂NH₂, SO₂NHC₁₋₆ alkyl, SO₂NHC₃₋₆ cycloalkyl SO₂N(C₁₋₆ alkyl)₂, SO₂N(C₃₋₆ cycloalkyl)₂, NHSO₂C₁₋₆ alkyl, NHSO₂C₃₋₆ cycloalkyl, CO₂C₁₋₆ alkyl, CO₂C₃₋₆ cycloalkyl, CONHC₁₋₆ alkyl, CONHC₃₋₆ cycloalkyl, CON(C₁₋₆ alkyl)₂, CON(C₃₋₆ cycloalkyl)₂OH, OC₁₋₃ alkyl, C₁₋₃ fluorinatedalkyl, OC₃₋₆ cycloalkyl, OC₃₋₆ cycloalkyl-C₁₋₃ alkyl, SH, SOₓC₁₋₃ alkyl, C₃₋₆ cycloalkyl, or SOₓC₃₋₆ cycloalkyl-C₁₋₃ alkyl, where x is 0, 1, or 2.

“Heteroaryl” refers to mono and bicyclic aromatic groups of 4 to 10 atoms containing at least one heteroatom. Heteroatom as used in the term heteroaryl refers to oxygen, sulfur and nitrogen. Examples of monocyclic heteroaryls include, but are not limited to, oxazinyl, thiazinyl, diazinyl, triazinyl, tetrazinyl, imidazolyl, tetrazolyl, isoxazolyl, furanyl, furazanyl, oxazolyl, thiazolyl, thiophenyl, pyrazolyl, triazolyl, and pyrimidinyl. Examples of bicyclic heteroaryls include but are not limited to, benzimidazolyl, indolyl, isoquinolinyl, indazolyl, quinolinyl, quinazolinyl, purinyl, benzisoxazolyl, benzoxazolyl, benzthiazolyl, benzodiazolyl, benzotriazolyl, isoindolyl and indazolyl. A heteroaryl group can be unsubstituted or substituted with one or more of the following groups: halogen, CN, OH, aryl, arylalkyl, heteroaryl, heteroarylalkyl, C₁₋₆ alkyl, C₂₋₆ alkenyl, C₂₋₆ alkynyl, C₁₋₃ fluorinatedalkyl, C₃₋₆ cycloalkyl, C₃₋₆cycloalkyl-C₁₋₃alkyl, NO₂, NH₂, NHC₁₋₆ alkyl, N(C₁₋₆ alkyl)₂, NHC₃₋₆ cycloalkyl, N(C₃₋₆ cycloalkyl)₂, NHC(O)C₁₋₆ alkyl, NHC(O)C₃₋₆ cycloalkyl, NHC(O)NHC₁₋₆ alkyl, NHC(O)NHC₃₋₆ cycloalkyl, SO₂NH₂, SO₂NHC₁₋₆ alkyl, SO₂NHC₃₋₆ cycloalkyl SO₂N(C₁₋₆ alkyl)₂, SO₂N(C₃₋₆ cycloalkyl)₂, NHSO₂C₁₋₆ alkyl, NHSO₂C₃₋₆ cycloalkyl, CO₂C₁₋₆ alkyl, CO₂C₃₋₆ cycloalkyl, CONHC₁₋₆ alkyl, CONHC₃₋₆ cycloalkyl, CON(C₁₋₆ alkyl)₂, CON(C₃₋₆ cycloalkyl)₂OH, OC₁₋₃ alkyl, C₁₋₃ fluorinatedalkyl, OC₃₋₆ cycloalkyl, OC₃₋₆ cycloalkyl-C₁₋₃ alkyl, SH, SOₓC₁₋₃ alkyl, C₃₋₆ cycloalkyl, or SOₓC₃₋₆ cycloalkyl-C₁₋₃ alkyl, where x is 0, 1, or 2.

“Arylalkyl” refers to an aryl group with at least one alkyl substitution. Examples of arylalkyl include, but are not limited to, toluenyl, phenylethyl, xylenyl, phenylbutyl, phenylpentyl, and ethylnaphthyl. An arylalkyl group can be unsubstituted or substituted with one or more of the following groups: H, halogen, CN, OH, aryl, arylalkyl, heteroaryl, heteroarylalkyl, C₁₋₆ alkyl, C₂₋₆ alkenyl, C₂₋₆ alkynyl, C₁₋₃ fluorinatedalkyl, C₃₋₆ cycloalkyl, C₃₋₆cycloalkyl-C₁₋₃alkyl, NO₂, NH₂, NHC₁₋₆ alkyl, N(C₁₋₆ alkyl)₂, NHC₃₋₆ cycloalkyl, N(C₃₋₆ cycloalkyl)₂, NHC(O)C₁₋₆ alkyl, NHC(O)C₃₋₆ cycloalkyl, NHC(O)NHC₁₋₆alkyl, NHC(O)NHC₃₋₆ cycloalkyl, SO₂NH₂, SO₂NHC₁₋₆ alkyl, SO₂NHC₃₋₆ cycloalkyl SO₂N(C₁₋₆ alkyl)₂, SO₂N(C₃₋₆ cycloalkyl)₂, NHSO₂C₁₋₆ alkyl, NHSO₂C₃₋₆ cycloalkyl, CO₂C₁₋₆ alkyl, CO₂C₃₋₆ cycloalkyl, CONHC₁₋₆ alkyl, CONHC₃₋₆ cycloalkyl, CON(C₁₋₆ alkyl)₂, CON(C₃₋₆ cycloalkyl)₂OH, OC₁₋₃ alkyl, C₁₋₃ fluorinatedalkyl, OC₃₋₆ cycloalkyl, OC₃₋₆ cycloalkyl-C₁₋₃ alkyl, SH, SOₓC₁₋₃ alkyl, C₃₋₆ cycloalkyl, or SOₓC₃₋₆ cycloalkyl-C₁₋₃ alkyl, where x is 0, 1, or 2.

“Heteroarylalkyl” refers to a heteroaryl group with at least one alkyl substitution. A heteroarylalkyl group can be unsubstituted or substituted with one or more of the following: H, halogen, CN, OH, aryl, arylalkyl, heteroaryl, heteroarylalkyl, C₁₋₆ alkyl, C₂₋₆ alkenyl, C₂₋₆ alkynyl, C₁₋₃ fluorinatedalkyl, C₃₋₆ cycloalkyl, C₃₋₆cycloalkyl-C₁₋₃alkyl, NO₂, NH₂, NHC₁₋₆ alkyl, N(C₁₋₆ alkyl)₂, NHC₃₋₆ cycloalkyl, N(C₃₋₆ cycloalkyl)₂, NHC(O)C₁₋₆ alkyl, NHC(O)C₃₋₆ cycloalkyl, NHC(O)NHC₁₋₆ alkyl, NHC(O)NHC₃₋₆ cycloalkyl, SO₂NH₂, SO₂NHC₁₋₆ alkyl, SO₂NHC₃₋₆ cycloalkyl SO₂N(C₁₋₆ alkyl)₂, SO₂N(C₃₋₆ cycloalkyl)₂, NHSO₂C₁₋₆ alkyl, NHSO₂C₃₋₆ cycloalkyl, CO₂C₁₋₆ alkyl, CO₂C₃₋₆ cycloalkyl, CONHC₁₋₆ alkyl, CONHC₃₋₆ cycloalkyl, CON(C₁₋₆ alkyl)₂, CON(C₃₋₆ cycloalkyl)₂OH, OC₁₋₃ alkyl, C₁₋₃ fluorinatedalkyl, OC₃₋₆ cycloalkyl, OC₃₋₆ cycloalkyl-C₁₋₃ alkyl, SH, SOₓC₁₋₃ alkyl, C₃₋₆ cycloalkyl, or SOₓC₃₋₆ cycloalkyl-C₁₋₃ alkyl, where x is 0, 1, or 2.

“C₁-C₆ alkyl” or “C₁₋₆ alkyl” refers to a straight or branched chain saturated hydrocarbon containing 1-6 carbon atoms. Examples of a C₁-C₆ alkyl group include, but are not limited to, methyl, ethyl, propyl, isopropyl, n-pentyl, isopentyl, neopentyl, and hexyl.

“C₂-C₆ alkenyl” or “C₂₋₆ alkenyl” refers to a straight or branched chain unsaturated hydrocarbon containing 2-6 carbon atoms and at least one double bond. Examples of a C₂-C₆ alkenyl group include, but are not limited to, ethylene, propylene, 1-butylene, 2-butylene, isobutylene, sec-butylene, 1-pentene, 2-pentene, isopentene, 1-hexene, 2-hexene, 3-hexene, and isohexene.

“C₃-C₆ alkenyl” or “C₃₋₆ alkenyl” refers to a straight or branched chain unsaturated hydrocarbon containing 3-6 carbon atoms and at least one double bond. Examples of a C₃-C₆ alkenyl group include, but are not limited to, propylene, 1-butylene, 2-butylene, isobutylene, sec-butylene, 1-pentene, 2-pentene, isopentene, 1-hexene, 2-hexene, 3-hexene, and isohexene.

“C₂-C₆ alkynyl” or “C₂₋₆ alkynyl” refers to a straight or branched chain unsaturated hydrocarbon containing 2-6 carbon atoms and at least one triple bond. Examples of a C₂-C₆ alkynyl group include, but are not limited to, acetylene, propyne, 1-butyne, 2-butyne, isobutyne, sec-butyne, 1-pentyne, 2-pentyne, isopentyne, 1-hexyne, 2-hexyne, and 3-hexyne.

“C₃-C₆ alkynyl” or “C₃₋₆ alkynyl” refers to a straight or branched chain unsaturated hydrocarbon containing 3-6 carbon atoms and at least one triple bond. Examples of a C₃-C₆ alkynyl group include, but are not limited to, propyne, 1-butyne, 2-butyne, isobutyne, sec-butyne, 1-pentyne, 2-pentyne, isopentyne, 1-hexyne, 2-hexyne, and 3-hexyne.

“C₁-C₆ alkoxy” or “C₁₋₆ alkoxy” refers to a straight or branched chain saturated or unsaturated hydrocarbon containing 1-6 carbon atoms and at least one oxygen atom. Examples of a C₁-C₆-alkoxy include, but are not limited to, methoxy, ethoxy, isopropoxy, butoxy, n-pentoxy, isopentoxy, neopentoxy, and hexoxy.

“C₃-C₆ cycloalkyl” or “C₃₋₆ cycloalkyl” refers to a cyclic saturated hydrocarbon containing 3-6 carbon atoms. Examples of a C₃-C₆ cycloalkyl group include, but are not limited to, cyclopropane, cyclobutane, cyclopentane, and cyclohexane.

“C₃-C₈ cycloalkenyl” or “C₃₋₈ cycloalkenyl” refers to a cyclic hydrocarbon containing 3-8 carbon atoms and a double bond. Examples of a C₃-C₈ cycloalkyl group include, but are not limited to, cyclobutene, cyclopentene, cyclohexene, and bicycloheptenes such as bicyclo[2.2.1]hept-2-ene.

“C₃-C₆ cycloalkyl-C₁-C₃ alkyl” or “C₃₋₆ cycloalkyl-C₁₋₃ alkyl” refers to a cyclic saturated hydrocarbon containing 3-6 carbon atoms that is further substituted with a straight or branched chain hydrocarbon containing 1-3 carbon atoms. Examples of a C₃-C₆ cycloalkyl-C₁-C₃ alkyl group include, but are not limited to, propylcyclopropane, propylcyclobutane, ethylcyclopropane, propylcyclopentane, and methylcyclohexane.

“C₁-C₃ fluorinated alkyl” or “C₁₋₃ fluorinated alkyl” refers to an saturated straight or branched chain hydrocarbon containing 1-3 carbon atoms that can be further substituted with other functional groups. Examples of a C₁-C₃ fluorinated alkyl are trifluoromethyl, 1,1,1-trifluoroethyl, and trifluoroacetyl.

A “subject” is a mammal, e.g., a human, mouse, rat, guinea pig, dog, cat, horse, cow, pig, or non-human primate, such as a monkey, chimpanzee, or baboon.

Representative “pharmaceutically acceptable salts” include, e.g., water-soluble and water-insoluble salts, such as the acetate, amsonate (4,4-diaminostilbene-2,2-disulfonate), benzenesulfonate, benzoate, bicarbonate, bisulfate, bitartrate, borate, bromide, butyrate, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucaamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts.

An “effective amount” when used in connection an pyrimidine sulfonamide analog is an amount effective for treating or preventing a disease associated with the canonical Wnt-β-catenin cellular messaging system.

The following abbreviations are used herein and have the indicated definitions: ACN is acetonitrile, HOAc is acetic acid, n-BuLi is normal butyl lithium, n-BuOH is normal butanol, DBU is 1,8-diazabicyclo[5.4.0]undec-7-ene, DDQ is 2,3-dicyano-5,6-dichloro-parabenzoquinone, DMA is dimethylacetamide, DMF is N,N-dimethylformamide, DMAP is 4-dimethylaminopyridine, DMSO is dimethylsulfoxide, EtOAc is ethyl acetate, EtOH is ethanol, FBS is fetal bovine serum, HPLC is high pressure liquid chromatography, LC/MS is liquid chromatography/mass spectroscopy, MeCN is acetonitrile, MeOH is methanol, MS is mass spectrometry, NaOAc is sodium acetate, NBS is N-bromosuccinimide, NMP is N-methyl-2-pyrrolidone, NMR is nuclear magnetic resonance, RP-HPLC is reverse phase high performance liquid chromatography, RPMI is Roswell Park Memorial Institute, T-BuOK is potassium tert-Butoxide, TEA is triethanolamine, THF is tetrahydrofuran, TFA is trifluoroacetic acid, TLC is thin-layer chromatography, p-TsOH is para-toluene sulfonic acid, p-TsCl is para-toluene sulfonyl chloride, and VLUX is a device for measuring luminescence.

### The Pyrimidine Sulfonamide Analogs of Formula (I)

The present invention provides pyrimidine sulfonamide analogs according to Formula (I) below:

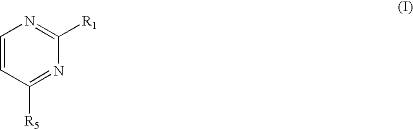

and pharmaceutically acceptable salts thereof,
wherein

R₅ is as defined above; and

R₁ is

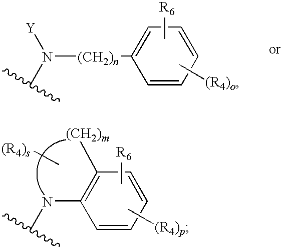

wherein
n, m, o, p, s, Y, R₄, and R₆ are as defined above.

In one embodiment, R₁ is

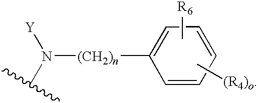

In one embodiment, n is 0.

In one embodiment, n is 1.

In one embodiment, R₂ and R₃ are H.

In one embodiment, R₁ is

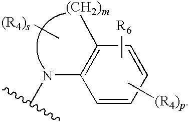

In one embodiment, m is 2.

In one embodiment, m is 3.

In one embodiment, R₂ and R₃ are H.

The invention also relates to compounds of Formula (II):

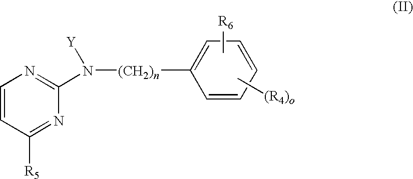

and pharmaceutically acceptable salts thereof,
wherein

R₄, R₅, R₆, n, o, and Y are as defined above.

In one embodiment, Y is H.

In one embodiment, n is 0.

In one embodiment, R₂ and R₃ are H.

Illustrative compounds of Formula II are exemplified by the following:
4-{[4-(3-Methylthien-2-yl)pyrimidin-2-yl]amino}benzenesulfonamide,
3-({4-[4-(Methylsulfonyl)phenyl]pyrimidin-2-yl}amino)benzenesulfonamide,
N-1,3-Thiazol-2-yl-4-[(4-thien-2-ylpyrimidin-2-yl)amino]benzenesulfonamide,
N-Methyl-4-{[4-(1,3-thiazol-2-yl)pyrimidin-2-yl]amino}benzenesulfonamide,
2-Methyl-N-pyrimidin-2-yl-4-{[4-(1,3-thiazol-2-yl)pyrimidin-2-yl]amino}benzenesulfonamide,
4-{[4-(1-Benzothien-2-yl)pyrimidin-2-yl]amino}-N-methyl-N-1,3-thiazol-2-ylbenzenesulfonamide,
2-Methyl-N-pyrimidin-2-yl-4-[(4-thien-3-ylpyrimidin-2-yl)amino]benzenesulfonamide,
N-Isobutyl-4-[(4-pyridin-4-ylpyrimidin-2-yl)amino]benzenesulfonamide,
2-Methyl-4-[(4-pyridin-4-ylpyrimidin-2-yl)amino]-N-pyrimidin-2-ylbenzenesulfonamide,
N-(4-{[2-(Methoxymethyl)pyrrolidin-1-yl]sulfonyl}phenyl)-4-pyridin-4-ylpyrimidin-2-amine,
2-Methyl-N-pyrimidin-2-yl-4-[(4-thien-2-ylpyrimidin-2-yl)amino]benzenesulfonamide,
N-(1-phenyl-1H-pyrazol-5-yl)-4-[(4-thien-2-ylpyrimidin-2-yl)amino]benzenesulfonamide,
N-methyl-N-1,3-thiazol-2-yl-4-[(4-thien-2-ylpyrilidin-2-yl)amino]benzenesulfonamide,
2-methyl-4-{[4-(1-methyl-1H-pyrrol-2-yl)pyrimidin-2-yl]amino}-N-pyrimidin-2-ylbenzenesulfonamide,
N-methyl-4-{[4-(1-methyl-1H-pyrrol-2-yl)pyrimidin-2-yl]amino}-N-1,3-thiazol-2-ylbenzenesulfonamide,
N-[4-(dimethylamino)phenyl]-4-[(4-pyridin-3-ylpyrimidin-2-yl)amino]benzenesulfonamide,
N-methyl-4-[(4-pyridin-3-ylpyrimidin-2-yl)amino]benzenesulfonamide,
2-methyl-4-[(4-pyridin-3-ylpyrimidin-2-yl)amino]-N-pyrimidin-2-ylbenzenesulfonamide,
N-(1-phenyl-1H-pyrazol-5-yl)-4-[(4-pyridin-3-ylpyrimidin-2-yl)amino]benzenesulfonamide,
N-(4-{[2-(methoxymethyl)pyrrolidin-1-yl]sulfonyl}phenyl)-4-pyridin-3-ylpyrimidin-2-amine,
4-{[4-(5-bromothien-2-yl)pyrimidin-2-yl]amino}-N-methyl-N-1,3-thiazol-2-ylbenzenesulfonamide,
N-methyl-4-{[4-(1-naphthyl)pyrimidin-2-yl]amino}-N-1,3-thiazol-2-ylbenzenesulfonamide,
N-(4-{[2-(methoxymethyl)pyrrolidin-1-yl]sulfonyl}phenyl)-4-(1-naphthyl)pyrimidin-2-amine,
N-methyl-4-{[4-(3-methylthien-2-yl)pyrimidin-2-yl]amino}-N-1,3-thiazol-2-ylbenzenesulfonamide,
2-{[(4-{[4-(3-methylthien-2-yl)pyrimidin-2-yl]amino}phenyl)sulfonyl]amino}-1,3-thiazole-4-carboxylic acid,
4-{[(4-pyridin-3-ylpyrimidin-2-yl)amino]methyl}benzenesulfonamide,
4-{[(4-pyridin-4-ylpyrimidin-2-yl)amino]methyl}benzenesulfonamide,
4-{[4-(2-thienyl)pyrimidin-2-yl]amino}methyl)benzenesulfonamide,
3-{[4-(1,3-thiazol-2-yl)pyrimidin-2-yl]amino}benzenesulfonamide,
4-[[4-(1-benzothien-2-yl)pyrimidin-2-yl](methyl)amino]benzenesulfonamide,
4-[[4-(1-benzothien-2-yl)pyrimidin-2-yl](methyl)amino]-N-methylbenzenesulfonamide,
3-{[4-(1-benzothien-2-yl)pyrimidin-2-yl]amino}-N-methylbenzenesulfonamide,
3-{[4-(1-benzothien-2-yl)pyrimidin-2-yl]amino}-N-isobutylbenzenesulfonamide, and
4-(4-(naphthalen-2-yl)pyrimidin-2-ylamino)benzenesulfonamide.

The invention also relates to compounds of the formula (III):

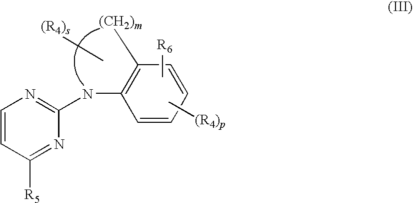

and pharmaceutically acceptable salts thereof,
wherein

R₄, R₅, R₆, m, and p are all defined as above.

In one embodiment, m is 2.

In one embodiment, m is 3.

Illustrative compounds of Formula II are exemplified by the following:
1-(4-(naphthalen-2-yl)pyrimidin-2-yl)-1,2,3,4-tetrahydroquinoline-6-sulfonamide,
1-(4-(thiophen-2-yl)pyrimidin-2-yl)indoline-5-sulfonamide,
1-(4-(naphthalen-2-yl)pyrimidin-2-yl)indoline-5-sulfonamide,
N,N-diethyl-1-(4-(thiophen-2-yl)pyrimidin-2-yl)indoline-5-sulfonamide,
N,N-diethyl-1-(4-(naphthalen-2-yl)pyrimidin-2-yl)indoline-5-sulfonamide,
N,N-dibenzyl-1-(4-(naphthalen-2-yl)pyrimidin-2-yl)indoline-5-sulfonamide,
1-(4-(thiophen-2-yl)pyrimidin-2-yl)-1,2,3,4-tetrahydroquinoline-6-sulfonamide, and
N,N-dimethyl-1-(4-(thiophen-2-yl)pyrimidin-2-yl)indoline-5-sulfonamide.

### Methods for Using Pyrimidine Sulfonamide Analogs

The pyrimidine sulfonamide analogs of the present invention exhibit agonism of the canonical Wnt-β-catenin cellular messaging system and, therefore, can be utilized in order to inhibit abnormal cell growth and/or encourage healthy cell regeneration or healthy cell growth. Thus, the pyrimidine sulfonamide analogs are effective in the treatment of disorders of the canonical Wnt-β-catenin cellular messaging system including, bone disorders, cancer, and Alzheimer's disease. In particular, the pyrimidine sulfonamide analogs of the present invention possess bone anabolic growth properties and have cancer cell growth inhibiting effects and are effective in treating cancers. Types of cancers that can be treated include but are not limited to solid cancers and malignant lymphomas, and also, leukemia, skin cancer, bladder cancer, breast cancer, uterus cancer, ovary cancer, prostate cancer, lung cancer, colon cancer, pancreas cancer, renal cancer, gastric cancer, brain tumor.

### Therapeutic Administration

When administered to a subject, the pyrimidine sulfonamide analogs or pharmaceutically acceptable salts thereof of the pyrimidine sulfonamide analogs can be administered neat or as a component of a composition that comprises a physiologically acceptable carrier or vehicle. A composition of the invention can be prepared using a method comprising admixing the pyrimidine sulfonamide analogs or a pharmaceutically acceptable salt of the pyrimidine sulfonamide analogs and a physiologically acceptable carrier, excipient, or diluent. Admixing can be accomplished using methods well known for admixing a pyrimidine sulfonamide analog or a pharmaceutically acceptable salt of the pyrimidine sulfonamide analog and a physiologically acceptable carrier, excipient, or diluent.

The present compositions, comprising pyrimidine sulfonamide analogs or pharmaceutically acceptable salts thereof of the pyrimidine sulfonamide analogs of the invention can be administered orally. The pyrimidine sulfonamide analogs or pharmaceutically acceptable salts thereof of pyrimidine sulfonamide analogs of the invention can also be administered by any other convenient route, for example, by infusion or bolus injection, by absorption through epithelial or mucocutaneous linings (e.g., oral, rectal, vaginal, and intestinal mucosa) and can be administered together with another therapeutic agent. Administration can be systemic or local. Various known delivery systems, including encapsulation in liposomes, microparticles, microcapsules, and capsules, can be used.

Methods of administration include, but are not limited to, enteral or parenteral administration such as intradermal, intramuscular, intraperitoneal, intravascular (e.g., intravenous or intra-arterial), subcutaneous, intranasal, epidural, oral, sublingual, intracerebral, intravaginal, intra-articular, intrathecal, transdermal, rectal, by inhalation, or topical, particularly to the ears, nose, eyes, or skin. In some instances, administration will result in release of the pyrimidine sulfonamide analog or a pharmaceutically acceptable salt of the pyrimidine sulfonamide analog into the bloodstream. The mode of administration is left to the discretion of the practitioner.

In one embodiment, the pyrimidine sulfonamide analog or a pharmaceutically acceptable salt of the pyrimidine sulfonamide analog is administered orally.

In another embodiment, the pyrimidine sulfonamide analog or a pharmaceutically acceptable salt of the pyrimidine sulfonamide analog is administered intravenously.

In another embodiment, the pyrimidine sulfonamide analog or a pharmaceutically acceptable salt of the pyrimidine sulfonamide analog can be administered locally. This can be achieved, for example, by local infusion during surgery, topical application, e.g., in conjunction with a wound dressing after surgery, by injection, by means of a catheter, by means of a suppository or edema, or by means of an implant, said implant being of a porous, non-porous, or gelatinous material, including membranes, such as sialastic membranes, or fibers.

In certain embodiments, the pyrimidine sulfonamide analog or a pharmaceutically acceptable salt of the pyrimidine sulfonamide analog can be introduced into the central nervous system, circulatory system or gastrointestinal tract by any suitable route, including intraventricular, intrathecal injection, paraspinal injection, epidural injection, enema, and by injection adjacent to the peripheral nerve. Intraventricular injection can be facilitated by an intraventricular catheter, for example, attached to a reservoir, such as an Ommaya reservoir.

Pulmonary administration can also be employed, e.g., by use of an inhaler or nebulizer, and formulation with an aerosolizing agent, or via perfusion in a fluorocarbon or synthetic pulmonary surfactant. In certain embodiments, the pyrimidine sulfonamide analog or a pharmaceutically acceptable salt of the pyrimidine sulfonamide analog can be formulated as a suppository, with traditional binders and excipients such as triglycerides.

In another embodiment, the pyrimidine sulfonamide analog or a pharmaceutically acceptable salt of the pyrimidine sulfonamide analog can be delivered in a vesicle, in particular a liposome (see Langer, Science 249:1527-1533 (1990) and Treat et al., Liposomes in the Therapy of Infectious Disease and Cancer pp. 317-327 and pp. 353-365 (1989)).

In yet another embodiment, the pyrimidine sulfonamide analog or a pharmaceutically acceptable salt of the pyrimidine sulfonamide analog can be delivered in a controlled-release system or sustained-release system (see, e.g., Goodson, in Medical Applications of Controlled Release, vol. 2, pp. 115-138 (1984). Other controlled or sustained-release systems discussed in the review by Langer, Science 249:1527-1533 (1990), can be used. In one embodiment, a pump can be used (Langer, Science 249:1527-1533 (1990); Sefton, CRC Crit. Ref. Biomed. Eng. 14:201 (1987); Buchwald et al., Surgery 88:507 (1980); and Saudek et al., N. Engl. J. Med. 321:574 (1989), the disclosures of which are herein incorporated by reference). In another embodiment, polymeric materials can be used (see Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 2:61 (1983); Levy et al., Science 228:190 (1935); During et al., Ann. Neural. 25:351 (1989); and Howard et al., J. Neurosurg. 71:105 (1989)).

In yet another embodiment, a controlled- or sustained-release system can be placed in proximity of a target of the pyrimidine sulfonamide analog or a pharmaceutically acceptable salt of the pyrimidine sulfonamide analog, e.g., the reproductive organs, thus requiring only a fraction of the systemic dose.

The present compositions can optionally comprise a suitable amount of a physiologically acceptable excipient.

Such physiologically acceptable excipients can be liquids, such as water and oils, including those of petroleum, animal, vegetable, or synthetic origin, such as peanut oil, soybean oil, mineral oil, sesame oil and the like. The physiologically acceptable excipients can be saline, gum acacia, gelatin, starch paste, talc, keratin, colloidal silica, urea and the like. In addition, auxiliary, stabilizing, thickening, lubricating, and coloring agents can be used. In one embodiment, the physiologically acceptable excipients are sterile when administered to an subject. The physiologically acceptable excipient should be stable under the conditions of manufacture and storage and should be preserved against the contaminating action of microorganisms. Water is a particularly useful excipient when the pyrimidine sulfonamide analog or a pharmaceutically acceptable salt of the pyrimidine sulfonamide analog is administered intravenously. Saline solutions and aqueous dextrose and glycerol solutions can also be employed as liquid excipients, particularly for injectable solutions. Suitable physiologically acceptable excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

Liquid carriers may be used in preparing solutions, suspensions, emulsions, syrups, and elixirs. The pyrimidine sulfonamide analog or pharmaceutically acceptable salt of the pyrimidine sulfonamide analog of this invention can be dissolved or suspended in a pharmaceutically acceptable liquid carrier such as water, an organic solvent, a mixture of both, or pharmaceutically acceptable oils or fat. The liquid carrier can contain other suitable pharmaceutical additives including solubilizers, emulsifiers, buffers, preservatives, sweeteners, flavoring agents, suspending agents, thickening agents, colors, viscosity regulators, stabilizers, or osmo-regulators. Suitable examples of liquid carriers for oral and parenteral administration include water (particular containing additives as above, e.g., cellulose derivatives, including sodium carboxymethyl cellulose solution), alcohols (including monohydric alcohols and polyhydric alcohols, e.g., glycols) and their derivatives, and oils (e.g., fractionated coconut oil and arachis oil). For parenteral administration the carrier can also be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellant.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the composition is in the form of a capsule. Other examples of suitable physiologically acceptable excipients are described in Remington's Pharmaceutical Sciences pp. 1447-1676 (Alfonso R. Gennaro, ed., 19th ed. 1995), the disclosure of which is herein incorporated by reference.

In one embodiment, the heteroaryl/aryl pyrimidine analog or a pharmaceutically acceptable salt of the heteroaryl/aryl pyrimidine analog is formulated in accordance with routine procedures as a composition adapted for oral administration to humans. Compositions for oral delivery can be in the form of, for example, tablets, lozenges, buccal forms, troches, aqueous or oily suspensions or solutions, granules, powders, emulsions, capsules, syrups, or elixirs. Orally administered compositions can contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. In powders, the carrier can be a finely divided solid, which is an admixture with the finely divided pyrimidine sulfonamide analog or pharmaceutically acceptable salt of the pyrimidine sulfonamide analog. In tablets, the pyrimidine sulfonamide analog or pharmaceutically acceptable salt of the pyrimidine sulfonamide analog is mixed with a carrier having the necessary compression properties in suitable proportions and compacted in the shape and size desired. The powders and tablets can contain up to about 99% of the pyrimidine sulfonamide analog or pharmaceutically acceptable salt of the pyrimidine sulfonamide analog.

Capsules may contain mixtures of the pyrimidine sulfonamide analogs or pharmaceutically acceptable salts thereof of the pyrimidine sulfonamide analogs with inert fillers and/or diluents such as pharmaceutically acceptable starches (e.g., corn, potato, or tapioca starch), sugars, artificial sweetening agents, powdered celluloses (such as crystalline and microcrystalline celluloses), flours, gelatins, gums, etc.

Tablet formulations can be made by conventional compression, wet granulation, or dry granulation methods and utilize pharmaceutically acceptable diluents, binding agents, lubricants, disintegrants, surface modifying agents (including surfactants), suspending or stabilizing agents (including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins. Surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidal silicon dioxide, phosphates, sodium dodecylsulfate, magnesium aluminum silicate, and triethanolamine.

Moreover, when in a tablet or pill form, the compositions can be coated to delay disintegration and absorption in the gastrointestinal tract, thereby providing a sustained action over an extended period of time. Selectively permeable membranes surrounding an osmotically active driving compound or a pharmaceutically acceptable salt of the compound are also suitable for orally administered compositions. In these latter platforms, fluid from the environment surrounding the capsule can be imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used. Oral compositions can include standard excipients such as mannitol, lactose, starch, magnesium stearate, sodium saccharin, cellulose, and magnesium carbonate. In one embodiment, the excipients are of pharmaceutical grade.

In another embodiment, the pyrimidine sulfonamide analog or a pharmaceutically acceptable salt of the pyrimidine sulfonamide analog can be formulated for intravenous administration. Typically, compositions for intravenous administration comprise sterile isotonic aqueous buffer. Where necessary, the compositions can also include a solubilizing agent. Compositions for intravenous administration can optionally include a local anesthetic such as lignocaine to lessen pain at the site of the injection. Generally, the ingredients are supplied either separately or mixed together in unit dosage form, for example, as a dry lyophilized powder or water-free concentrate in a hermetically sealed container such as an ampule or sachette indicating the quantity of active agent. Where the pyrimidine sulfonamide analog or a pharmaceutically acceptable salt of the pyrimidine sulfonamide analog is to be administered by infusion, it can be dispensed, for example, with an infusion bottle containing sterile pharmaceutical grade water or saline. Where the pyrimidine sulfonamide analog or a pharmaceutically acceptable salt of the pyrimidine sulfonamide analog is administered by injection, an ampule of sterile water for injection or saline can be provided so that the ingredients can be mixed prior to administration.

In another embodiment, the pyrimidine sulfonamide analog or pharmaceutically acceptable salt of the pyrimidine sulfonamide analog can be administered transdermally through the use of a transdermal patch. Transdermal administrations include administrations across the surface of the body and the inner linings of the bodily passages including epithelial and mucosal tissues. Such administrations can be carried out using the present pyrimidine sulfonamide analogs or pharmaceutically acceptable salts thereof of the pyrimidine sulfonamide analogs, in lotions, creams, foams, patches, suspensions, solutions, and suppositories (e.g., rectal or vaginal).

Transdermal administration can be accomplished through the use of a transdermal patch containing the pyrimidine sulfonamide analog or pharmaceutically acceptable salt of the pyrimidine sulfonamide analog and a carrier that is inert to the pyrimidine sulfonamide analog or pharmaceutically acceptable salt of the pyrimidine sulfonamide analog, is non-toxic to the skin, and allows delivery of the agent for systemic absorption into the blood stream via the skin. The carrier may take any number of forms such as creams or ointments, pastes, gels, or occlusive devices. The creams or ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the pyrimidine sulfonamide analog or pharmaceutically acceptable salt of the pyrimidine sulfonamide analog into the blood stream, such as a semi-permeable membrane covering a reservoir containing the pyrimidine sulfonamide analog or pharmaceutically acceptable salt of the pyrimidine sulfonamide analog with or without a carrier, or a matrix containing the active ingredient.

The pyrimidine sulfonamide analogs or pharmaceutically acceptable salts thereof of the pyrimidine sulfonamide analogs of the invention may be administered rectally or vaginally in the form of a conventional suppository. Suppository formulations may be made from traditional materials, including cocoa butter, with or without the addition of waxes to alter the suppository's melting point, and glycerin. Water-soluble suppository bases, such as polyethylene glycols of various molecular weights, may also be used.

The pyrimidine sulfonamide analog or a pharmaceutically acceptable salt of the pyrimidine sulfonamide analog can be administered by controlled-release or sustained-release means or by delivery devices that are known to those of ordinary skill in the art. Such dosage forms can be used to provide controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled- or sustained-release. Advantages of controlled- or sustained-release compositions include extended activity of the drug, reduced dosage frequency, and increased compliance by the subject being treated. In addition, controlled- or sustained-release compositions can favorably affect the time of onset of action or other characteristics, such as blood levels of the pyrimidine sulfonamide analog or a pharmaceutically acceptable salt of the pyrimidine sulfonamide analog, and can thus reduce the occurrence of adverse side effects.

Controlled- or sustained-release compositions can initially release an amount of the pyrimidine sulfonamide analog or a pharmaceutically acceptable salt of the pyrimidine sulfonamide analog that promptly produces the desired therapeutic or prophylactic effect, and gradually and continually release other amounts of the pyrimidine sulfonamide analog or a pharmaceutically acceptable salt of the pyrimidine sulfonamide analog to maintain this level of therapeutic or prophylactic effect over an extended period of time. To maintain a constant level of the pyrimidine sulfonamide analog or a pharmaceutically acceptable salt of the pyrimidine sulfonamide analog in the body, the pyrimidine sulfonamide analog or a pharmaceutically acceptable salt of the pyrimidine sulfonamide analog can be released from the dosage form at a rate that will replace the amount of the pyrimidine sulfonamide analog or a pharmaceutically acceptable salt of the pyrimidine sulfonamide analog being metabolized and excreted from the body. Controlled- or sustained-release of an active ingredient can be stimulated by various conditions, including but not limited to, changes in pH, changes in temperature, concentration or availability of enzymes, concentration or availability of water, or other physiological conditions.

The amount of the pyrimidine sulfonamide analog or a pharmaceutically acceptable salt of the pyrimidine sulfonamide analog that is effective for treating or preventing a canonical Wnt-β-catenin cellular messaging system-related disorder can be determined using standard clinical techniques. In addition, in vitro or in vivo assays can optionally be employed to help identify suitable dosage ranges. The precise dose to be employed can also depend on the route of administration, the condition, the seriousness of the condition being treated, as well as various physical factors related to the individual being treated, and can be decided according to the judgment of an ordinarily skilled health-care practitioner. The typical dose will range from about 0.001 mg/kg to about 250 mg/kg of body weight per day. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The number and frequency of dosages corresponding to a completed course of therapy can be readily determined according to the judgment of an ordinarily skilled health-care practitioner; that is, if more than one pyrimidine sulfonamide analog or a pharmaceutically acceptable salt of the pyrimidine sulfonamide analog is administered, the effective dosage amounts correspond to the total amount administered.

In one embodiment, the pharmaceutical composition is in unit dosage form, e.g., as a tablet, capsule, powder, solution, suspension, emulsion, granule, or suppository. In such form, the composition is sub-divided in unit dose containing appropriate quantities of the active ingredient; the unit dosage form can be packaged compositions, for example, packeted powders, vials, ampoules, pre-filled syringes or sachets containing liquids. The unit dosage form can be, for example, a capsule or tablet itself, or it can be the appropriate number of any such compositions in package form. Such unit dosage form may contain from about 1 mg/kg to about 250 mg/kg, and may be given in a single dose or in two or more divided doses.

The pyrimidine sulfonamide analog or a pharmaceutically acceptable salt of the pyrimidine sulfonamide analog can be assayed in vitro or in vivo for the desired therapeutic or prophylactic activity prior to use in humans. Animal model systems can be used to demonstrate safety and efficacy.

The present methods for treating or preventing a canonical Wnt-β-catenin cellular messaging system-related disorder, can further comprise administering another therapeutic agent to the subject being administered the pyrimidine sulfonamide analog or a pharmaceutically acceptable salt of the pyrimidine sulfonamide analog.

Effective amounts of the other therapeutic agents are well known to those skilled in the art. However, it is well within the skilled artisan's purview to determine the other therapeutic agent's optimal effective amount range. The pyrimidine sulfonamide analog or a pharmaceutically acceptable salt of the pyrimidine sulfonamide analog and the other therapeutic agent can act additively or, in one embodiment, synergistically. In one embodiment, of the invention, where another therapeutic agent is administered to an subject, the effective amount of the pyrimidine sulfonamide analog or a pharmaceutically acceptable salt of the pyrimidine sulfonamide analog is less than its effective amount would be where the other therapeutic agent is not administered. In this case, without being bound by theory, it is believed that the pyrimidine sulfonamide analog or a pharmaceutically acceptable salt of the pyrimidine sulfonamide analog and the other therapeutic agent act synergistically.

Suitable other therapeutic agents useful in the methods and compositions of the present invention include, but are not limited to, cancer agents, Alzheimer's agents, bone disorder agents, osteoporosis agents, rheumatoid arthritis agents, osteoarthritis agents, and hormone replacement agents.

Suitable cancer agents useful in the methods and compositions of the present invention include, but are not limited to, temozolomide, a topoisomerase I inhibitor, procarbazine, dacarbazine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epirubicin, 5-fluorouracil, taxanes such as docetaxel and paclitaxel, leucovorin, levamisole, irinotecan, estramustine, etoposide, nitrogen mustards, 1,3-bis(2-chloroethyl)-1-nitrosourea (BCNU), nitrosoureas such as carmustine and lomustine, vinca alkaloids such as vinblastine, vincristine and vinorelbine, platinum complexes such as cisplatin, carboplatin and oxaliplatin, imatinib mesylate, hexamethylmelamine, topotecan, tyrosine kinase inhibitors, tyrphostins herbimycin A, genistein, erbstatin, and lavendustin A.

Other therapeutic agents useful in the methods and compositions of the present invention include, but are not limited to, hydroxyzine, glatiramer acetate, interferon beta-1a, interferon beta-1b, mitoxantrone, and natalizumab.

Suitable Alzheimer's agents useful in the methods and compositions of the present invention include, but are not limited, to donepezil, galantamine, memantine, niacin, rivastigmine, and tacrine.

Suitable bone disorder and/or osteoporosis agents useful in the methods and compositions of the present invention include, but are not limited, to alendronate, bazedoxifene, calcitonin, clomifene, lasofoxifene, ormeloxifene, raloxifene, tamoxifen, and toremifene.

Suitable rheumatoid arthritis agents useful in the methods and compositions of the present invention include, but are not limited to, abatacept, acetaminophen adalimumab, aspirin, auranofin, azathioprine, celecoxib, cyclophosphamide, cyclosporine, diclofenac, etanercept, hydroxychloroquine, ibuprofen, indomethacin, infliximab, ketoprofen, leflunomide, methotrexate, minocycline, nabumetone, naproxen, rituximab, and sulfasalazine.

Suitable osteoarthritis agents useful in the methods and compositions of the present invention include, but are not limited to, acetaminophen, aspirin, celecoxib, cortisone, hyaluronic acid, ibuprofen, nabumetone, naproxen, rofecoxib, and valdecoxib.

Suitable hormone replacement therapy agents useful in the methods and compositions of the present invention include, but are not limited to, estrogen, estradiol, medroxyprogesterone, norethindrone, and progesterone.

In one embodiment, the pyrimidine sulfonamide analog or a pharmaceutically acceptable salt of the pyrimidine sulfonamide analog is administered concurrently with another therapeutic agent.

In one embodiment, a composition comprising an effective amount of the pyrimidine sulfonamide analog or a pharmaceutically acceptable salt of the pyrimidine sulfonamide analog and an effective amount of another therapeutic agent within the same composition can be administered.

In another embodiment, a composition comprising an effective amount of the pyrimidine sulfonamide analog or a pharmaceutically acceptable salt of the pyrimidine sulfonamide analog and a separate composition comprising an effective amount of another therapeutic agent can be concurrently administered.

In another embodiment, an effective amount of the pyrimidine sulfonamide analog or a pharmaceutically acceptable salt of the pyrimidine sulfonamide analog is administered prior to or subsequent to administration of an effective amount of another therapeutic agent. In this embodiment, the pyrimidine sulfonamide analog or a pharmaceutically acceptable salt of the pyrimidine sulfonamide analog is administered while the other therapeutic agent exerts its therapeutic effect, or the other therapeutic agent is administered while the pyrimidine sulfonamide analog or a pharmaceutically acceptable salt of the pyrimidine sulfonamide analog exerts its preventative or therapeutic effect for treating or preventing a canonical Wnt-β-catenin cellular messaging system-related disorder.

In another embodiment, the pharmaceutically acceptable carrier is suitable for oral administration and the composition comprises an oral dosage form.

The pyrimidine sulfonamide analogs and pharmaceutically acceptable salts thereof of pyrimidine sulfonamide analogs can be prepared using a variety of methods starting from commercially available compounds, known compounds, or compounds prepared by known methods. General synthetic routes to many of the compounds of the invention are included in the following schemes. It is understood by those skilled in the art that protection and deprotection steps not shown in the Schemes may be required for these syntheses, and that the order of steps may be changed to accommodate functionality in the target molecule.

Methods useful for making the pyrimidine sulfonamide analogs are set forth in the Examples below and generalized in Schemes.

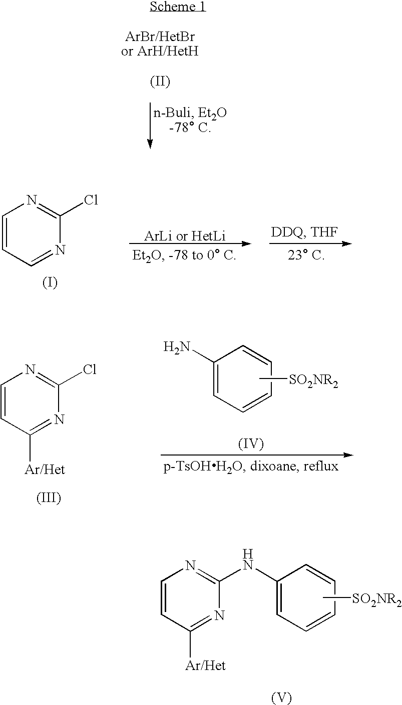

wherein R₂ is as defined above.

Compounds of the formula (V) may be prepared according to Scheme 1 by reacting 2-chloropyrimidine (I) with aryl or heteroaryllithiums, prepared by reacting aryl bromides/heteroaryl bromides (II) with a strong base such as n-BuLi, MeLi or PhLi or via deprotonation of aryls/heteroaryls (II) with a strong base such as n-BuLi, MeLi, PhLi, LDA, or LiN(TMS)₂, followed by oxidation with DDQ to give 4-aryl/heteroaryl-2-chloropyrimidines (III) according to the procedures of Czarny and Harden. (Strekowski, L et al., J. Heterocyclic. Chem. 1990, 27, 1393, and Harden D. B. et al., J. Org. Chem. 1988, 53, 4137). Alternatively, the 4-aryl-2-chloropyrimidine intermediate can be prepared by treating the corresponding arylacetyl compound III-A with DMF dimethylacetyl to provide the vinylogous amide III-B. Further treatment of III-B with urea provides the pyrimidinone product III-C which is converted to the chloride III after refluxing in phosphorous oxychloride for several hours (see e.g. WO 2005/049581).

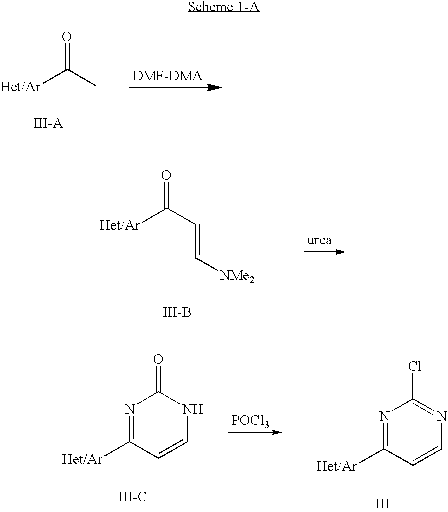

A subsequent reaction with anilino sulfonamides (IV) in hot dioxane in the presence of p-TsOH.H₂O gives the desired 2-aminopyrimidine sulfonamides (V) based on the procedure of Hattinger (Hattinger, G. et al., GB 2369359).

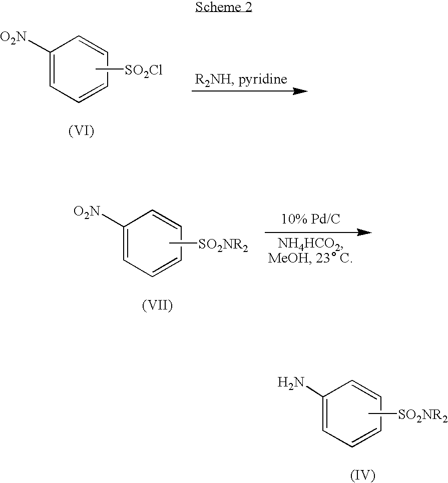

wherein R₂ is as defined above.

Compounds of the formula (IV) can be purchased commercially or prepared via the procedure in Scheme 2: nitrobenzenesulfonyl chlorides (VI) may be converted to the corresponding sulfonamides (VII) via reaction with R₂NH in an amine solvent such as pyridine or in a polar aprotic solvent such as CH₂Cl₂ or THF in the presence of a hindered amine base such as iPr₂NEt or Et₃N and DMAP, these nitrobenzenesulfonamides (VII) may be reduced to the corresponding amines using conditions such as 10% Pd/C, NH₄HCO₂, MeOH, or SnCl₂-H₂O, EtOH, heat or Fe, HCl, EtOH, H₂O, heat.

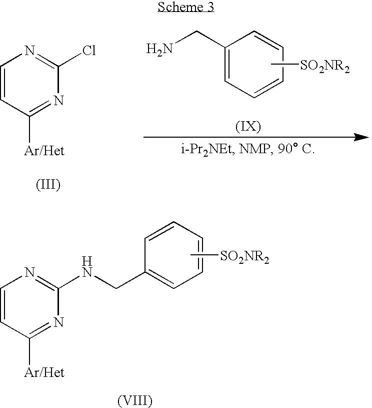

wherein R₂ is as defined above.

Compounds of formula (VIII) may be prepared according to Scheme 3. Thus 4-aryl/heteroaryl-2-chloropyrimidines (III) are combined with benzylaminosulfonamides (IX) in a polar aprotic solvent such as NMP, DMSO or DMF and a hindered base such as iPr₂NEt, Et₃N, t-BuOK or DBU according to the procedure of Kindon (Kindon, N. et al. WO 9902501) to give compounds of structure (VIII).

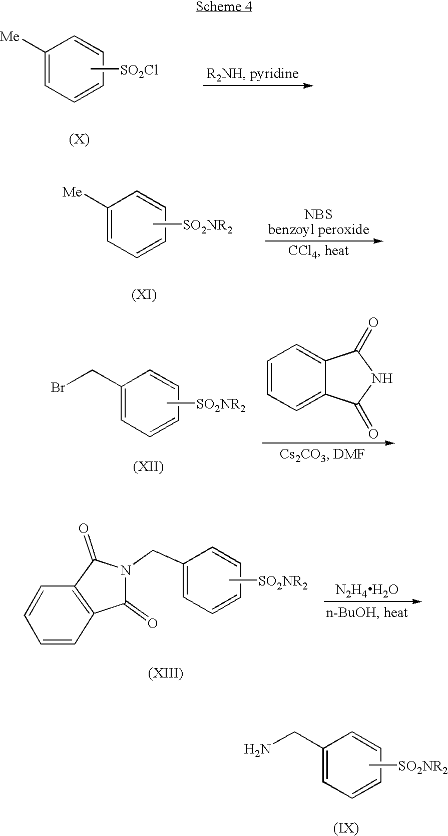

wherein R₂ is as defined above.

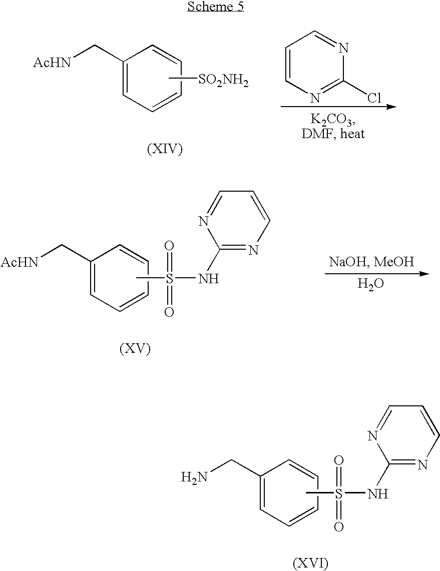

wherein R₂ is as defined above.

Compounds of formula (IX) may be prepared according to Schemes 4 and 5. Thus p-TsCl (X) may be reacted with various amines in an amine base such as pyridine according to the procedure of Hamdouchi (Hamdouchi, C. et al. J. Med. Chem. 2003, 46, 4333, the disclosure of which is herein incorporated by reference) to give the corresponding sulfonamides (XI). Reaction of these sulfonamides with NBS, benzoyl peroxide initiator in hot CCl₄ gives the corresponding benzyl bromides (XII). Conversion to the corresponding phthalimides (XIII) occurs using phthalimide, Cs₂CO₃ in a polar aprotic solvent such as DMF, DMSO, or NMP. Deprotection to give the corresponding benzylaminosulfonamides (IX) occurs using hydrazine monohydrate in hot n-BuOH.

Alternatively, aromatic sulfonamides (XVI) may be prepared according to the procedure outlined in Scheme 5. Thus benzylaminosulfonamides (XIV) may be alkylated with aromatic chlorides and fluorides in a polar aprotic solvent such as DMF, DMSO or NMP according to the procedure of Matsukawa (Matsukawa, O. et al. Chem. Abstr. 1951, 8994) to yield compounds such as the N-acetyl-2-aminopyrimidine (XV). Hydrolysis of the acetyl moiety yields the desired aromatic sulfonamide (XVI).

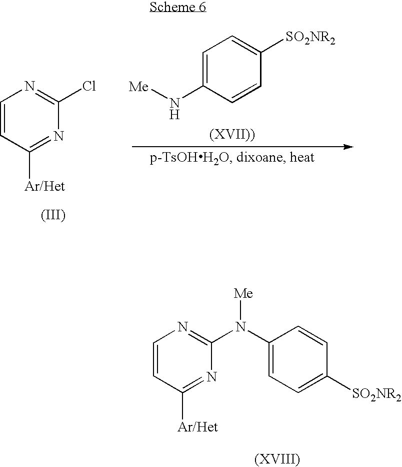

wherein R₂ is as defined above.

Compounds of formula (XVIII) may be prepared according to the procedure outlined in Scheme 6. Thus 4-aryl/heteroaryl-2-chloropyrimidines (III) are combined with 4-methylaminobenzene sulfonamides (XVII) in hot dioxane in the presence of p-TsOH.H₂O to give the desired N-methylaminosulfonamide sulfonamides (XVIII).

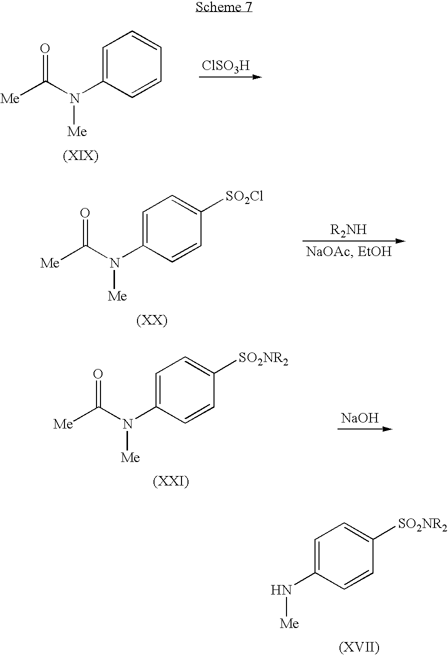

wherein R₂ is as defined above.

Compounds of formula (XVII) are prepared according to Scheme 7. Thus N-methyl acetamide (XIX) may be converted to sulfonyl chloride (XX) according to the procedure of Stojanovic (Stojanovic, O. K. et al. Chem. Abstr. 1973, 3902) using neat ClSO₃H. Conversion to the corresponding sulfonamides (XXI) using amines, NaOAc in EtOH and NaOH hydrolysis of the acetyl group to produce the desired 4-methylaminobenzene sulfonamides (XVII) was performed according to the procedure of Oinuma (Oinuma, H. et al. J. Med. Chem. 1991, 34, 2260).

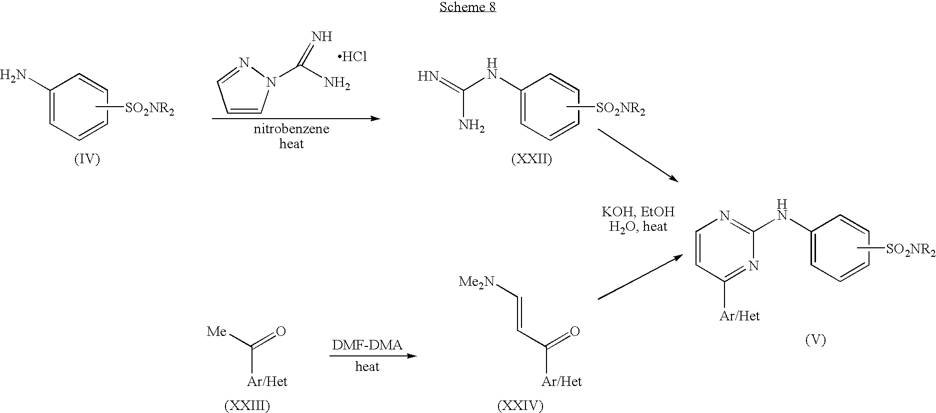

wherein R₂ is as defined above.

Compounds of formula (V) may also be prepared according to Scheme 8 using the procedure first outlined by Bredereck (Bredereck, H. et al. Ber., Dtsch. Chem. Ges. 1964, 97, 3397). Thus anilines (IV) may be converted to the corresponding aryl guanidines using pyrazole-1-carboxamidine according to the procedure of Bernatowicz (Bernatowicz, M. S. et al. J. Org. Chem. 1992, 57, 2497). The guanidines may be combined with 3-dimethylamino-1-aryl/heteroaryl-propenones (XXIV), prepared according to the procedure of (X) by heating methyl ketones (XXIII) with DMF DMA, in the presence of a base such as KOH, NaOH, or Et₃N or acid such as HOAc in hot EtOH or MeOH to give the desired 2-aminopyrimidines (V).

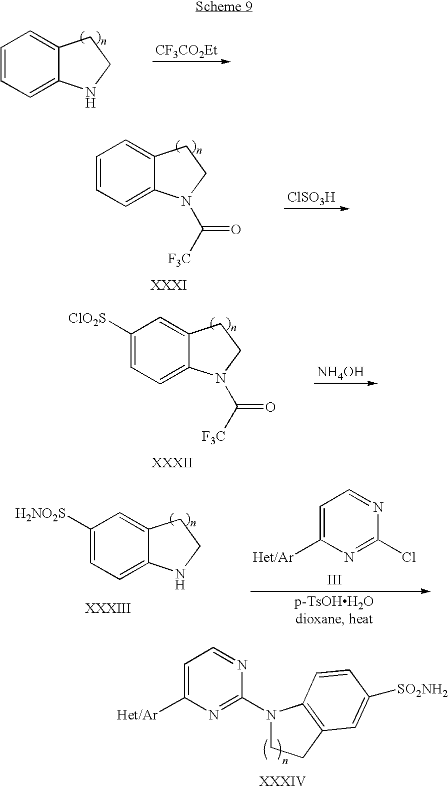

n is 1 or 2.

Compounds of the formula XXXIV were prepared according to Scheme 9. Protection of indoline or tetrahydroquinoline as the trifluoroacetamide provides intermediates such as XXXI which undergo chlorosulfonylation as described by Mathvink (Mathvink, R. J. et al. Bioorg. and Med. Chem. Letters, 1999, 9, 1869) to afford sulfonyl chlorides XXXII. Treatment with ammonium hydroxide provides the deprotected sulfonamides XXXIII, which react with 2-chloropyrimidines III to afford cyclic sulfonamides such as XXXIV.

I. General Experimental for the Preparation of 2-anilino-4-aryl/heteroarylpyrimidine Primary Sulfonamides (Procedure A)

### A. Step 1: Preparation of 2-chloro-4-aryl/heteroaryl-pyrimidine

To a ⁻30° C. solution of a Ar/HetLi (10.66 mmol, 1.08 eq, generated via deprotonation of Li for Br exchange) in 20 ml of Et₂O is added portion wise a suspension of 2-chloropyrimidine (9.84 mmol, 1 equiv.) in 20 ml Et₂O in 2 ml portions over 15 min. The resulting suspension is stirred for 30 min. at ⁻30° C. and at 0° C. for 60 min. The reaction is quenched with H₂O (0.27 ml, 1.5 equiv.) in THF (3 ml) and DDQ (2.95 g, 10.66 mmol, 1 equiv.) and THF (15 ml) is then added. The resulting suspension was stirred at 23° C. for 15 min., and then cooled to 0° C. Hexanes (10 ml) are added followed by 0° C. solution of NaOH (10 ml, 3N). The suspension is stirred for 5 min. at 0° C., 100 ml of H₂O is added and the layers are separated. The organic layer is dried (Na₂SO₄) and concentrated in vacuo. Purification via SiO₂ gel column chromatography gives the title compound.

### B. Step 2: Preparation of 2-anilino-4-aryl/heteroarylpyrimidine primary sulfonamides

A 2-chloro-4-aryl/heteroaryl pyrimidine (0.26 mmol, 1 equiv.), aniline (0.26 mmol, 1 equiv.), and 1,4-dioxane (2 mL) solution is combined with a solution of p-TsOH (0.21 mmol, 0.8 eq) and 1,4-dioxane (1 ml). The resulting suspension is heated at 100° C. for 12-18 h. Reaction progress is monitored using an analytical HP Agilent 1100 LC/MS.

### HPLC: Analytical Method and Parameters:

### Instrument: HP Agilent 1100 LC/MS

### UV Detector: Agilent 1100 Diode Array Detector

### Mass Spectrometer Detector: Agilent MSD

Column: Waters Xterra MS C18 30 mm×2.1 mm i.d., 3.5 um
Flow Rate: 1.00 ml/min.

### Run Time: 5.00 min.

Gradient Elution: 0 min. 90% water, 10% acetonitrile; 3 min. 10% water, 90% acetonitrile

### Column Temperature: 50° C.

### UV Signals: 215 nm, 254 nm

### MS Parameters: Mass Range 100-1000, Fragmentor 140, Gain EMV 1.0

After cooling to 23° C., all volatiles are removed in a Speed Vac. This crude material is dissolved in 0.5 ml DMSO: 1.5 ml MeCN, filtered through a 0.45 μm GMF, and purified on a Gilson HPLC, using a Phenomenex LUNA C₁₈ column: 60 mm×21.20 mm I.D., 5 μm particle size: with ACN/water (containing 0.2% TFA or Et₃N) gradient elution. The appropriate fractions are analyzed by LC/MS as described above. Combining pure fractions and evaporating the solvent in a Speed Vac isolates the title compound.

II. General Experimental for the Preparation of 2-anilino-4-aryl/heteroarylpyrimidine Primary Sulfonamides (Procedure B)

### A. Step 1: Preparation of 3-dimethylamino-1-aryl/heteroaryl-propenone

A 0.1 M solution of a methyl ketone is heated at 130° C. for 12 h. After cooling to 23° C., all volatiles are evaporated. The remaining material is dissolved in a minimum of CH₂Cl₂ and passed through as short SPE SiO₂ gel cartridge eluting with additional CH₂Cl₂. The eluant is concentrated to a minimum volume and equal amount of hexanes was added. Cooling to 5° C. produces crystals of the title compound as a yellow or orange solid.

### B. Step 2: Preparation of 2-anilino-4-aryl/heteroarylpyrimidine Primary Sulfonamides

Aniline (1 equiv.) is combined with 1.5 equiv. of 1H-pyrazole-1-carboxamidine hydrochloride as a 0.1 M nitrobenzene solution and is heated to 200° C. for 6 h. After cooling to 23° C., 1 equiv. of 3-dimethylamino-1-aryl/heteroaryl-propenone is added followed by 1.25 equiv. of KOH, EtOH (equal volume to that of nitrobenzene) and H₂O, ( 1/10^{th} the volume of EtOH). This mixture is heated at 120° C. for 12 h, cooled to 23° C. and evaporated in a Speed-Vac. This crude material is dissolved in 0.5 ml DMSO:1.5 ml MeCN, filtered through a 0.45 μm GMF, and purified on a Gilson HPLC, using a Phenomenex LUNA C₁₈ column: 60 mm×21.20 mm I.D., 5 μm particle size: with ACN/water (containing 0.2% TFA or Et₃ N) gradient elution. The appropriate fractions are analyzed by LC/MS as described above. Combining pure fractions and evaporating the solvent in a Speed-Vac isolates the title compound.

The following compounds were prepared according to Procedure B:

| | | LC Retention | ESMS | |
|---|---|---|---|---|
| Example | Name | Time (min)^{a} | Ion^{b} | Procedure^{a} |
| 13 | 4-{[4-(3-Methylthien-2-yl)pyrimidin-2- | 2.73 | 347 (M + H)⁺ | A |
| | yl]amino}benzenesulfonamide | | | |
| 32 | 3-({4-[4-(Methylsulfonyl)phenyl]pyrimidin-2- | — | 405 (M + H)⁺ | A |
| | yl}amino)benzenesulfonamide | | | |
| 69 | 3-{[4-(1,3-thiazol-2-yl)pyrimidin-2- | 1.86 | 334 (M + H)⁺ | A |
| | yl]amino}benzenesulfonamide | | | |

^{a}HPLC Conditions: Instrument - Agilent 1100; Column: Keystone Aquasil C18 (as above); Mobile Phase
A: 10 mM NH₄OAC in 95% water/5% CAN; Mobile Phase B: 10 mM NH₄OAC in 5% water/95%
CAN; Flow Rate: 0.800 ml/min.; Column Temperature: 40° C.; Injection Volume: 5 μl; UV: monitor 215,
230, 254, 280, and 300 nm; Purity is reported at 254 nm unless otherwise noted.
Gradient Table:

| | |
|---|---|
| Time (min.) | % B |
| 0.0 | 0 |
| 2.5 | 100 |
| 4.0 | 100 |
| 4.1 | 0 |
| 5.5 | 0 |

^{b}MS Conditions: Instrument: Agilent MSD; Ionization Mode: API-ES; Gas Temperature: 350° C.; Drying
Gas: 11.0 L/min..; Nebulizer Pressure: 55 psig; Polarity: 50% positive, 50% negative; VCap: 3000 V
(positive), 2500 V (negative); Fragmentor: 80 (positive), 120 (negative); Mass Range: 100-1000 m/z;
Threshold: 150; Step size: 0.15; Gain: 1; Peak width: 0.15 min.

III. General Experimental for the Preparation of 2-anilino-4-aryl/heteroarylpyrimidine sulfonamide Secondary and Tertiary Sulfonamides

### A. Step 1: Preparation of Substituted-4-nitro-benzenesulfonamides

To 1 equiv. of 4-nitrobenzenesulfonyl chloride as a 0.1 M solution in CH₂Cl₂ is added 1.25 eq of i-Pr₂NEt, 0.1 equiv. of DMAP and 1.25 equiv. of amine. This mixture is stirred at 23° C. until judged complete by TLC. After quenching with sat. NaHCO₃ solution and separation of the organic and aqueous layers, the organic layer is evaporated to yield nearly pure 4-nitrobenzenesulfonamides as off-white to colorless solids (Yield range: 56-100% yields).

### B. Step 2: Preparation of 4-amino-benzenesulfonamide Secondary and Tertiary Sulfonamides

To 1 eq of a 4-nitrobenzenesulfonamide as a 0.1 M solution in MeOH is added 0.1 wt. equiv. of 10% Pd/C and 5 equiv. of ammonium formate and the mixture is stirred at 23° C. for 8 h. Filtration through celite and evaporation gives the title compound as an off-white solid or a colorless oil.

### C. Step 3: Preparation of 2-anilino-4-aryl/heteroarylpyrimidine sulfonamide Secondary and Tertiary Sulfonamides

The reaction conditions outlined above in Procedure A are used substituting 4-amino-benzenesulfonamide secondary and tertiary sulfonamides for the 2-anilino-4-aryl/heteroarylpyrimidine primary sulfonamides.

The following compounds were prepared according to the indicated procedure:

| | | LC Retention | |
|---|---|---|---|
| Example | Name | Time (min)^{a} | ESMS Ion^{b} |
| 33 | N-1,3-Thiazol-2-yl-4-[(4-thien-2-ylpyrimidin-2- | — | 416 (M + H)⁺ |
| | yl)amino]benzenesulfonamide | | |
| 34 | N-Methyl-4-{[4-(1,3-thiazol-2-yl)pyrimidin-2- | — | 348 (M + H)⁺ |
| | yl]amino}benzenesulfonamide | | |
| 35 | 2-Methyl-N-pyrimidin-2-yl-4-{[4-(1,3-thiazol-2- | — | 426 (M + H)⁺ |
| | yl)pyrimidin-2-yl]amino}benzenesulfonamide | | |
| 36 | 4-{[4-(1-Benzothien-2-yl)pyrimidin-2- | — | 480 (M + H)⁺ |
| | yl]amino}-N-methyl-N-1,3-thiazol-2- | | |
| | ylbenzenesulfonamide | | |
| 38 | 2-Methyl-N-pyrimidin-2-yl-4-[(4-thien-3- | 2.16 | 425 (M + H)⁺ |
| | ylpyrimidin-2-yl)amino]benzenesulfonamide | | |
| 39 | N-Isobutyl-4-[(4-pyridin-4-ylpyrimidin-2- | 2.22 | 384 (M + H)⁺ |
| | yl)amino]benzenesulfonamide | | |
| 40 | 2-Methyl-4-[(4-pyridin-4-ylpyrimidin-2- | 1.90 | 420 (M + H)⁺ |
| | yl)amino]-N-pyrimidin-2-ylbenzenesulfonamide | | |
| 41 | N-(4-{[2-(Methoxymethyl)pyrrolidin-1- | 2.22 | 422 (M + H)⁺ |
| | yl]sulfonyl}phenyl)-4-pyridin-4-ylpyrimidin-2- | | |
| | amine | | |
| 44 | 2-Methyl-N-pyrimidin-2-yl-4-[(4-thien-2- | — | 425 (M + H)⁺ |
| | ylpyrimidin-2-yl)amino]benzenesulfonamide | | |
| 45 | N-(1-phenyl-1H-pyrazol-5-yl)-4-[(4-thien-2- | — | 475 (M + H)⁺ |
| | ylpyrimidin-2-yl)amino]benzenesulfonamide | | |
| 46 | N-methyl-N-1,3-thiazol-2-yl-4-[(4-thien-2- | — | 430 (M + H)⁺ |
| | ylpyrimidin-2-yl)amino]benzenesulfonamide | | |
| 50 | 2-methyl-4-{[4-(1-methyl-1H-pyrrol-2- | — | 422 (M + H)⁺ |
| | yl)pyrimidin-2-yl]amino}-N-pyrimidin-2- | | |
| | ylbenzenesulfonamide | | |
| 51 | N-methyl-4-{[4-(1-methyl-1H-pyrrol-2- | — | 427 (M + H)⁺ |
| | yl)pyrimidin-2-yl]amino}-N-1,3-thiazol-2- | | |
| | ylbenzenesulfonamide | | |
| 52 | N-[4-(dimethylamino)phenyl]-4-[(4-pyridin-3- | 2.21 | 447 (M + H)⁺ |
| | ylpyrimidin-2-yl)amino]benzenesulfonamide | | |
| 53 | N-methyl-4-[(4-pyridin-3-ylpyrimidin-2- | 1.92 | 342 (M + H)⁺ |
| | yl)amino]benzenesulfonamide | | |
| 54 | 2-methyl-4-[(4-pyridin-3-ylpyrimidin-2- | 1.88 | 420 (M + H)⁺ |
| | yl)amino]-N-pyrimidin-2-ylbenzenesulfonamide | | |
| 55 | N-(1-phenyl-1H-pyrazol-5-yl)-4-[(4-pyridin-3- | 1.95 | 470 (M + H)⁺ |
| | ylpyrimidin-2-yl)amino]benzenesulfonamide | | |
| 56 | N-(4-{[2-(methoxymethyl)pyrrolidin-1- | 2.22 | 426 (M + H)⁺ |
| | yl]sulfonyl}phenyl)-4-pyridin-3-ylpyrimidin-2- | | |
| | amine | | |
| 58 | 4-{[4-(5-bromothien-2-yl)pyrimidin-2- | 2.69 | 508 (M + H)⁺ |
| | yl]amino}-N-methyl-N-1,3-thiazol-2- | | |
| | ylbenzenesulfonamide | | |
| 60 | N-methyl-4-{[4-(1-naphthyl)pyrimidin-2- | 2.63 | 474 (M + H)⁺ |
| | yl]amino}-N-1,3-thiazol-2- | | |
| | ylbenzenesulfonamide | | |
| 61 | N-(4-{[2-(methoxymethyl)pyrrolidin-1- | 2.58 | 475 (M + H)⁺ |
| | yl]sulfonyl}phenyl)-4-(1-naphthyl)pyrimidin-2- | | |
| | amine | | |
| 64 | N-methyl-4-{[4-(3-methylthien-2-yl)pyrimidin- | 2.55 | 444 (M + H)⁺ |
| | 2-yl]amino}-N-1,3-thiazol-2- | | |
| | ylbenzenesulfonamide | | |
| 65 | 2-{[(4-{[4-(3-methylthien-2-yl)pyrimidin-2- | 1.72 | 474 (M + H)⁺ |
| | yl]amino}phenyl)sulfonyl]amino}-1,3-thiazole- | | |
| | 4-carboxylic acid | | |
| 78 | 3-{[4-(1-benzothien-2-yl)pyrimidin-2-yl]amino}- | 2.45 | 397 (M + H)⁺ |
| | N-methylbenzenesulfonamide | | |
| 80 | 3-{[4-(1-benzothien-2-yl)pyrimidin-2-yl]amino}- | 2.64 | 439 (M + H)⁺ |
| | N-isobutylbenzenesulfonamide | | |

^{a}HPLC Conditions: Instrument - Agilent 1100; Column: Keystone Aquasil C18 (as
above); Mobile Phase A: 10 mM NH₄OAC in 95% water/5% CAN; Mobile Phase B: 10 mM
NH₄OAC in 5% water/95% CAN; Flow Rate: 0.800 ml/min.; Column Temperature:
40° C.; Injection Volume: 5 ul; UV: monitor 215, 230, 254, 280, and 300 nm; Purity is
reported at 254 nm unless otherwise noted.
Gradient Table:

| | |
|---|---|
| Time (min.) | % B |
| 0.0 | 0 |
| 2.5 | 100 |
| 4.0 | 100 |
| 4.1 | 0 |
| 5.6 | 0 |

^{b}MS Conditions: Instrument: Agilent MSD; Ionization Mode: API-ES; Gas Temperature:
350° C.; Drying Gas: 11.0 L/min.; Nebulizer Pressure: 55psig; Polarity: 50% positive, 50%
negative; VCap: 3000 V (positive), 2500 V (negative); Fragmentor: 80 (positive), 120
(negative); Mass Range: 100-1000 m/z; Threshold: 150; Step size: 0.15; Gain: 1; Peak
width: 0.15 min.

IV. General Experimental for the Preparation of 2-benzylamino-4-aryl/heteroarylpyrimidine Primary Sulfonamides

To 0.1 M NMP solution of 1 equiv. of 2-chloro-4-aryl/heteroaryl-pyrimidine is added 1.2 equiv. of 4-aminomethyl-benzenesulfonamide and 5 equiv. of i-Pr₂NEt. After heating at 90° C. for 12 h, the reaction is cooled to 23° C. and 10 ml of H₂O was added. The mixture is extracted with 3×5 ml of ethyl acetate, and the combined organics are washed with 4×5 ml of H₂O, and evaporated to yield an orange viscous oil. Purification by RP-HPLC as outlined in Procedure A, Step 2 gives the title compounds.

The following compounds were prepared according to the above procedure:

| | | LC Retention | ESMS |
|---|---|---|---|
| Example | Name | Time (min.)^{a} | Ion^{b} |
| 66 | 4-{[(4-pyridin-3-ylpyrimidin-2- | 1.75 | 342 (M + H)⁺ |
| | yl)amino]methyl}benzenesulfonamide | | |
| 67 | 4-{[(4-pyridin-4-ylpyrimidin-2- | 1.39 | 342 (M + H)⁺ |
| | yl)amino]methyl}benzenesulfonamide | | |
| 68 | 4-({[4-(2-thienyl)pyrimidin-2- | 1.74 | 347 (M + H)⁺ |
| | yl]amino}methyl)benzenesulfonamide | | |

^{a}HPLC Conditions: Instrument - Agilent 1100; Column: Keystone Aquasil C18 (as
above); Mobile Phase A: 10 mM NH₄OAC in 95% water/5% CAN; Mobile Phase B: 10 nM
NH₄OAC in 5% water/95% CAN; Flow Rate: 0.800 ml/min.; Column Temperature:
40° C.; Injection Volume: 5 ul; UV: monitor 215, 230, 254, 280, and 300 nm; Purity is
reported at 254 nm unless otherwise noted.
Gradient Table:

| | |
|---|---|
| Time (min.) | % B |
| 0.0 | 0 |
| 2.5 | 100 |
| 4.0 | 100 |
| 4.1 | 0 |
| 5.7 | 0 |

^{b}MS Conditions: Instrument: Agilent MSD; Ionization Mode: API-ES; Gas Temperature:
350° C.; Drying Gas: 11.0 L/min.; Nebulizer Pressure: 55psig; Polarity: 50% positive, 50%
negative; VCap: 3000 V (positive), 2500 V (negative); Fragmentor: 80 (positive), 120
(negative); Mass Range: 100-1000 m/z; Threshold: 150; Step size: 0.15; Gain: 1; Peak
width: 0.15 min.

V. General Experimental for the Preparation of 2-N(Me)-anilino-4-aryl/heteroarylpyrimidine sulfonamides

### A. Step 1: 4-(Acetyl-methyl-amino)-benzenesulfonyl chloride

N-Methyl-N-phenyl-acetamide (10.0 g, 67 mmol) is heated with 50 ml of ClSO₃H at 70° C. for 90 min. The mixture is poured into 200 ml of ice and the resulting product is filtered, and washed with 2×25 ml of H₂O to the give the title compound as an off-white solid. (Based on the procedure of O. K. Stojanovic et al. Chem. Abstr. 1973, 78, 3902 s).

### B. Step 2: N-Substituted-N-(4-sulfamoyl-phenyl)-acetamides

To a 0.1 M EtOH slurry of 1.1 equiv. of amine and 2.7 equiv. of NaOAc at 0° C. is added 1 equiv. of 4-(acetyl-methyl-amino)-benzenesulfonyl chloride. The mixture is allowed to stir at 23° C. for 6 h. Water is added, and the mixture is extracted with 3×25 ml of EtOAc. The combined organics are washed with 1×50 ml of H₂O and 1×50 ml brine, dried over MgSO₄, filtered and evaporated to give the title compound as an off-white solid or oil. (Based on the procedure of H. Oinuma et al. J. Med. Chem. 1991, 34, 2260-7).

### C. Step 3: 4-Methylamino-benzenesulfonamides

A N-substituted-N-(4-sulfamoyl-phenyl)-acetamide (1 equiv.) is combined with 1 N aqueous NaOH to make a 0.1 M solution in acetamide. The resulting mixture is refluxed for 12 h. After cooling to 23° C., the reaction mixture is adjusted to pH ˜7 with 1 N aqueous HCl, and extracted with 2×25 ml EtOAc. The combined organics are washed with 1×50 ml H₂O, 1×50 ml brine, dried over MgSO₄, filtered and evaporated to give the title compound as a colorless solid or oil.

### D. Step 4: 2-N(Me)-anilino-4-aryl/heteroarylpyrimidine sulfonamides

The protocol described in Procedure A, step 2 is used except that 4-methylamino-benzenesulfonamides are used in place of primary amino-benzenesulfonamides.

The following compounds were prepared according to the above procedure:

| | | LC Retention Time | ESMS |
|---|---|---|---|
| Example | Name | (min)^{a} | Ion^{b} |
| 72 | 4-[[4-(1-benzothien-2-yl)pyrimidin-2- | 2.49 | 397 (M + H)⁺ |
| | yl](methyl)amino]benzenesulfonamide | | |
| 75 | 4-[[4-(1-benzothien-2-yl)pyrimidin-2- | 2.49 | 411 (M + H)⁺ |
| | yl](methyl)amino]-N-methylbenzenesulfonamide | | |

^{a}HPLC Conditions: Instrument - Agilent 1100; Column: Keystone Aquasil C18 (as above);
Mobile Phase A: 10 mM NH₄OAC in 95% water/5% CAN; Mobile Phase B: 10 mM
NH₄OAC in 5% water/95% CAN; Flow Rate: 0.800 ml/min.; Column Temperature: 40° C.;
Injection Volume: 5 ul; UV: monitor 215, 230, 254, 280, and 300 nm; Purity is reported at
254 nm unless otherwise noted.
Gradient Table:

| | |
|---|---|
| Time (min.) | % B |
| 0.0 | 0 |
| 2.5 | 100 |
| 4.0 | 100 |
| 4.1 | 0 |
| 5.8 | 0 |

^{b}MS Conditions: Instrument: Agilent MSD; Ionization Mode: API-ES; Gas Temperature:
350° C.; Drying Gas: 11.0 L/min.; Nebulizer Pressure: 55psig; Polarity: 50% positive, 50%
negative; VCap: 3000 V (positive), 2500 V (negative); Fragmentor: 80 (positive), 120
(negative); Mass Range: 100-1000 m/z; Threshold: 150; Step size: 0.15; Gain: 1; Peak
width: 0.15 min.

### Preparation of indoline-5- and tetrahydroquinoline-6-sulfonamide Analogs (Procedure B)

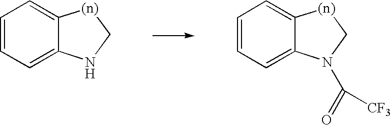

n is 1 or 2.

### Step 2-B: 1-(2,3-Dihydro-indol-1-yl)-2,2,2-trifluoro-ethanone (n=1)

Indoline (3 g, 25 mmol) is dissolved in MeOH (10 mL). Then, TEA (3.5 mL) and ethyl trifluoroacetate (7.1 g, 50 mmol) are added and the reaction mixture stirred for 4 h at 65° C. The mixture is concentrated and purified by ISCO (0% ethyl acetate/hexane-50% ethyl acetate) to yield 4.85 g (90.6%) of the title product. LC/MS [Column: Xterra MS C18, 5μ, 50×2.1 mm. Mobile phase: 90/10-5/95 water (0.1% formic acid)/acetonitrile (0.1% formic acid), 2 min., hold 1.5 min., 0.8 mL/min., 210-400 nm]: rt=1.79 min., calculated mass=215, [M+H]⁺=216.

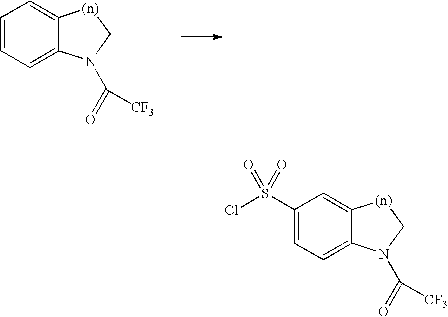

n is 1 or 2.

### Step 3-B: 1-(2,2,2-Trifluoro-acetyl)-2,3-dihydro-1H-indole-5-sulfonyl chloride (n=1)

Chlorosulfonic acid (6.5 g, 55.8 mmol) is added to the protected indoline prepared in step 2-B (4.0 g, 18.6 mmol) at 0° C., then warmed up to room temperature and stirred for 2 h. The reaction mixture is poured slowly into cold water and the product extracted with ethyl acetate (150 mL), dried under Na₂SO₃, evaporated and purified by ISCO (0% ethyl acetate/hexane-60% ethyl acetate) to yield 3.55 g (61%) of the title product. LC/MS [Column: Xterra MS C18, 5μ, 50×2.1 mm. Mobile phase: 90/10-5/95 water (0.1% formic acid)/acetonitrile (0.1% formic acid), 2 min., hold 1.5 min., 0.8 mL/min., 210-400 nm]: rt=1.92 min., calculated mass=313, [M-Cl+OH]⁻=294.

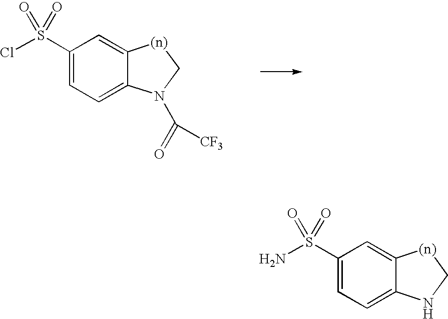

### Step 4-B: 2,3-Dihydro-1H-indole-5-sulfonic acid amide (n=1)

The indoline sulfonyl chloride from step 3-B (3 g, 9.58 mmol) is dissolved in dioxane (7 mL). Then NH₄OH [28-30%] (25 mL) is added and the reaction mixture stirred overnight. Water (100 mL) is added to the mixture and the product extracted with ethyl acetate (150 mL). The organic layer is dried over Na₂SO₃, and evaporated to yield 1.7 g (90%) of product. LC/MS [Column: Xterra MS C18, 5μ, 50×2.1 mm. Mobile phase: 90/10-5/95 water (0.1% formic acid)/acetonitrile (0.1% formic acid), 2 min., hold 1.5 min., 0.8 mL/min., 210-400 nm]: rt=0.51 min., calculated mass=198, [M+H]⁺=199.

### Step 4-B: Using the method of Step 7-A, replace 1,1-dioxo-2,3-dihydro-benzo[d]isothiazol-5-ylamine with 2,3-dihydro-1H-indole-5-sulfonic acid amide (n=1)

Step 2-C: Using the method of Step 7-A, replace 1,1-dioxo-2,3-dihydro-benzo[d]isothiazol-5-ylamine with sulfanilamide.

The following compounds were prepared according to the above procedures:

| | | | | HPLC R.t | HPLC |
|---|---|---|---|---|---|
| | Name | Procedure | MS | (min.) | Method |
| 82 | 1-(4-(naphthalen-2-yl)pyrimidin-2-yl)-1,2,3,4- | B | 417 | 10.5 | A |
| | tetrahydroquinoline-6-sulfonamide | | | | |
| 83 | 1-(4-(thiophen-2-yl)pyrimidin-2-yl)indoline- | B | 359 | 9.3 | A |
| | 5-sulfonamide | | | | |
| 84 | 1-(4-(naphthalen-2-yl)pyrimidin-2- | B | 376 | 10.8 | A |
| | yl)indoline-5-sulfonamide | | | | |
| 86 | 4-(4-(naphthalen-2-yl)pyrimidin-2- | C | 377 | 9.9 | A |
| | ylamino)benzenesulfonamide | | | | |
| 93 | 1-(4-(thiophen-2-yl)pyrimidin-2-yl)-1,2,3,4- | B | 373 | 9.2 | A |
| | tetrahydroquinoline-6-sulfonamide | | | | |

HPLC Methods: A: Column; Xterra RP18, 3.5μ, 150 × 4.6 mm. Mobile phase: 85/15-5/95 Ammonium formate buffer (pH = 3.5)/ACN + MeOH (1:1) for 10 min., hold 4 min., 1.2 mL/min., 210-370 nm. B: Column; Xterra RP18, 3.5μ, 150 × 4.6 mm. Mobile phase: 85/15-5/95 Ammonium bicarbonate buffer (pH = 9.5)/ACN + MeOH (1:1) for 10 min., hold 4 min., 1.2 mL/min., 210-370 nm.

Preparation of Dialkylated Cyclic Sulfonamides

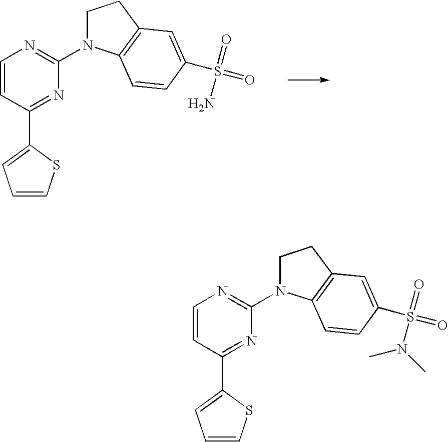

### 1-(3-Thiophen-2-yl-phenyl)-2,3-dihydro-1H-indole-5-sulfonic acid dimethylamine

1-(4-Thiophen-2-yl-pyrimidin-2-yl)-2,3-dihydro-1H-indole-5-sulfonic acid amide (15.6 mg, 43.6 mmol) is dissolved in DMSO (0.5 mL). To the vial is added NaH [60% dispersion in mineral oil] (9 mg, 218 mmol) followed by iodomethane (62 mg, 436 mmol). The reaction mixture is put on a shaker block for 14 h at 50° C. To the vial is added water (0.1 mL) and the solution purified using RP HPLC (YMC CombiPrep ProC18 50×20 mm I.D. column, S-5 μm, 12 nm. Flow rate 20 mL/min. Gradient: 10/90 Acetonitrile/Water (0.1% TFA in both solvents) to 100% acetonitrile over 10 minutes then hold for three minutes at 100% acetonitrile and ramp back to 10/90 acetonitrile/water over two minutes) and concentrated on a speed vac to afford the title compound (7.1 mg, 42%). LC/MS [Column: Waters Atlantis C18, 5μ, 4.6×150 mm. Mobile phase: 95/5-5/95 water (0.1% formic acid)/acetonitrile (0.1% formic acid), 6 min., hold 1.2 min., 1.5 mL/min., 210-400 nm], rt=3.2 min., purity=100%, calculated mass=386, [M+H]⁺=387.

The following compounds were prepared by the above procedure:

| | | | HPLC R.t | HPLC |
|---|---|---|---|---|
| No | Name | MS | (min.) | Method |
| 88 | N,N-diethyl-1-(4-(thiophen-2-yl)pyrimidin-2-yl)indoline-5- | 415 | 3.3 | A |
| | sulfonamide | | | |
| 89 | N,N-diethyl-1-(4-(naphthalen-2-yl)pyrimidin-2-yl)indoline- | 459 | 3.6 | A |
| | 5-sulfonamide | | | |
| 90 | N,N-dibenzyl-1-(4-(naphthalen-2-yl)pyrimidin-2- | 583 | 3.9 | A |
| | yl)indoline-5-sulfonamide | | | |

HPLC Methods: A: Column: Waters Atlantis C18, 5μ, 2 × 50 mm. Mobile phase: 95/5-5/95 water (10 mM ammonium acetate)/acetonitrile (10 mM ammonium acetate), 2.5 min., hold 1.5 min., 0.8 mL/min., 210-400 nm. B: Column; Xterra RP18, 3.5μ, 150 × 4.6 mm. Mobile phase: 85/15-5/95 Ammonium formate buffer (pH = 3.5)/ACN + MeOH (1:1) for 10 min., hold 4 min., 1.2 mL/min., 210-370 nm.

### Biological Evaluation—Functional Dkk1-LRP5-TCF-Luciferase Assay in U2OS Cells

U2OS Human Bone derived cells (Osteosarcoma) are grown in McCoy's SA Medium (Modified), with L-glutamine (GIBCO Cat No. 16600-082)+1% Pen-Strep+5% FBS) plated at 1×107 cells/T175 cm flask. The next day, the cells are co-transfected overnight with the following plasmids: (a) Test reporter (16xTCF-TK-FireFly-Luci), (b) Internal Control Reporter (TK-Renilla-Luci), (c) Wnt3a and (d) Dkk1. GIBCO's Lipofectamine 2000 and OptiMEM were used for the transfection. After a minimum of 4 hr of transfection at 37° C., the plasmid-transfected cells are trypsinized, counted, and suspended in freezing medium (95% FBS+5% DMSO). The reporter cells are frozen at 1×107/ml concentrations, aliquoted into 0.5 ml or 2.5 ml/tube and stored at 70° C.

The following day, test compounds are added under HTS setup by Plate Track into 384 well plates (white, TC treated, Falcon plate) such that the final concentration of the compounds in 20 μL/well cell will be 5 μg/ml (final concentration of DMSO=0.25% and final compound concentration=20 μM). Vials of frozen reporter cells are thawed by warming the vial in a 37° C. water bath for 60-120 seconds with some shaking until the cells formed a suspension. The thawed cells are transferred into a cold 50 ml (or larger) tube and mixed well by gentle pipetting. The appropriate amount of cold Phenol Red Free RPMI medium-1640 (GIBCO, Cat # 11835-030) with L-glutamine is added, both with ˜5% FBS (GIBCO-BRL, Cat. # 16000-044), so that 20 μl of the final cell suspension will contain ˜5,000 cells. The cell dilution is done such that the final concentration of FBS was ˜5%. Diluted cells (20 μl) are added into each well in a 384 well plate. The plate is incubated at 37° C. under 5% CO₂ for ˜20 h. Bright-Glo substrate, 2.5 μl/well is added, and the Fire Fly Luciferase is measured using VLUX (60 second exposure) immediately after the substrate was added. Test compounds are dissolved in DMSO (100%) and added to specified wells. Raw luciferase signal data obtained as relative luminescence units (RLUs) for the test compounds are normalized to the signal of the mean of the sample reporter cell plate with DMSO.

Active compounds have TCF-luciferase ratios of 2.5 fold or greater over DMSO. All compounds show a signal increase of at least 10% compared to a signal with only DMSO added.

While particular embodiments of the present invention have been illustrated and described, it would be apparent to those skilled in the art that various other changes and modifications can be made without departing from the spirit and scope of the invention. It is therefore intended to cover in the appended claims all such changes and modifications that are within the scope of this invention.

What is claimed is:

1. A compound of the Formula (I): 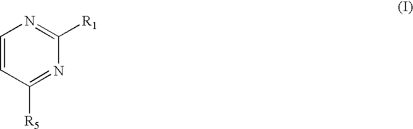
and pharmaceutically acceptable salts thereof,
wherein
R₅ is aryl, heteroaryl or C₄-C₈ cycloalkenyl both optionally substituted with 1-7 R₄ groups; and
R₁ is 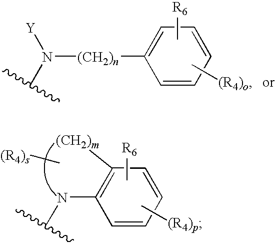
wherein
Y is H, C₁₋₆ alkyl, aryl, or arylalkyl;
R₆ is —SO₂NR₂R₃ or 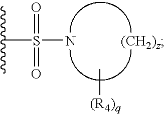
R₂ and R₃ are each independently H, C₁₋₆ alkyl, C₂₋₆ alkenyl, C₂₋₆ alkynyl, C₃₋₆cycloalkyl, C₃₋₆ cycloalkyl-C₁₋₃ alkyl, aryl, arylalkyl, heteroaryl, or heteroarylalkyl, whereas all except H can be optionally substituted with 1-5 R₄ groups;
each R₄ is independently H, halogen, CN, OH, aryl, arylalkyl, heteroaryl, heteroarylalkyl, C₁₋₆ alkyl, C₂₋₆ alkenyl optionally substituted with di(C₁-C₆)alkylaminocarbonyl or with di(C₁-C₆)alkylamino-(C₁-C₆)alkyloxycarbonyl, C₂₋₆ alkynyl optionally substituted with heteroaryl, C₁₋₃ fluorinatedalkyl, C₃₋₆ cycloalkyl, C₃₋₆ cycloalkyl-C₁₋₃ alkyl, NO₂, NH₂, NHC₁₋₆ alkyl, N(C₁₋₆ alkyl)₂, NHC₃₋₆ cycloalkyl, N(C₃₋₆ cycloalkyl)₂, NHC(O)C₁₋₆ alkyl, NHC(O)C₃₋₆ cycloalkyl, NHC(O)NHC₁₋₆ alkyl, NHC(O)NHC₃₋₆ cycloalkyl, SO₂NH₂, SO₂NHC₁₋₆ alkyl, SO₂NHC₃₋₆ cycloalkyl, SO₂N(C₁₋₆ alkyl)₂, SO₂N(aryl-C₁₋₆ alkyl)₂, SO₂N(C₃₋₆ cycloalkyl)₂, NHSO₂C₁₋₆ alkyl, NHSO₂C₃₋₆ cycloalkyl, CO₂H. CO₂C₁₋₆ alkyl, CO₂C₃₋₆ cycloalkyl, CONHC₁₋₆ alkyl, CONHC₃₋₆ cycloalkyl, CON(C₁₋₆ alkyl)₂, CON(C₃₋₆ cycloalkyl)₂OH, OC₁₋₃ alkyl, C₁₋₃ fluorinatedalkyl, OC₃₋₆ cycloalkyl, OC₃₋₆ cycloalkyl-C₁₋₃ alkyl, C₁₋₃alkyloxy-C₁₋₃ alkyl, SH, SOₓC₁₋₃ alkyl, C₃₋₆ cycloalkyl, or SOₓC₃₋₆ cycloalkyl-C₁₋₃ alkyl;
or if R₄ and R₆ are bonded to phenyl ring carbons that are adjacent to each other, then R₄ and R₆ taken together with the two phenyl ring carbons form a heteroaromatic ring containing an —SO₂—NH—, an —SO₂—N(C₁-C₆ alkyl)-, or an —SO₂—N(aryl)-;
n is 0 or 1;
m is 2 or 3;
o is 0, 1, 2, 3, or 4;
p is 0, 1, 2, 3, 4, 5, or 6;
q is 0, 1, 2, 3, 4, 5, 6, 7, 8, 9, 10, 11, or 12;
s is 0, 1, 2, 3, 4, 5, or 6;
x is 0, 1, or 2; and
z is 3, 4, 5, or 6.

2. The compound of claim 1 wherein R₁ is

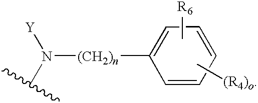

3. The compound of claim 2, wherein n is 0.

4. The compound of claim 2, wherein n is 1.

5. The compound of claim 2, wherein R₂ and R₃ are H.

6. The compound of claim 1, wherein R₁ is

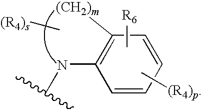

7. The compound of claim 6, wherein m is 2.

8. The compound of claim 6, wherein m is 3.

9. The compound of claim 6, wherein R₂ and R₃ are H.

10. A compound of the formula (II): 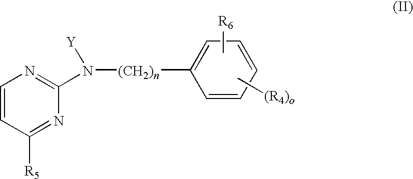
and pharmaceutically acceptable salts thereof,
wherein
R₅ is aryl or heteroaryl both optionally substituted with 1-7 R₄ groups;
Y is H, C₁₋₆ alkyl, aryl, or arylalkyl;
R₆ is —SO₂NR₂R₃ or 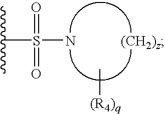
R₂ and R₃ are each independently H, C₁₋₆ alkyl, C₂₋₆ alkenyl, C₂₋₆ alkynyl, C₃₋₆ cycloalkyl, C₃₋₆ cycloalkyl-C₁₋₃ alkyl, aryl, arylalkyl, heteroaryl, or heteroarylalkyl, whereas all except H can be optionally substituted with 1-5 R₄ groups;
each R₄ is independently H, halogen, CN, OH, aryl, arylalkyl, heteroaryl, heteroarylalkyl, C₁₋₆ alkyl, C₂₋₆ alkenyl, C₂₋₆ alkynyl, C₁₋₃ fluorinatedalkyl, C₃₋₆ cycloalkyl, C₃₋₆ cycloalkyl-C₁₋₃ alkyl, NO₂, NH₂, NHC₁₋₆ alkyl, N(C₁₋₆ alkyl)₂, NHC₃₋₆ cycloalkyl, N(C₃₋₆ cycloalkyl)₂, NHC(O)C₁₋₆ alkyl, NHC(O)C₃₋₆ cycloalkyl, NHC(O)NHC₁₋₆ alkyl, NHC(O)NHC₃₋₆ cycloalkyl, SO₂NH₂, SO₂NHC₁₋₆ alkyl, SO₂NHC₃₋₆ cycloalkyl SO₂N(C₁₋₆ alkyl)₂, SO₂N(C₃₋₆ cycloalkyl)₂, NHSO₂C₁₋₆ alkyl, NHSO₂C₃₋₆ cycloalkyl, CO₂C₁₋₆ alkyl, CO₂C₃₋₆ cycloalkyl, CONHC₁₋₆ alkyl, CONHC₃₋₆ cycloalkyl, CON(C₁₋₆ alkyl)₂, CON(C₃₋₆ cycloalkyl)₂OH, OC₁₋₃ alkyl, C₁₋₃ fluorinatedalkyl, OC₃₋₆ cycloalkyl, OC₃₋₆ cycloalkyl-C₁₋₃ alkyl, SH, SOₓC₁₋₃ alkyl, C₃₋₆ cycloalkyl, or SOₓC₃₋₆ cycloalkyl-C₁₋₃ alkyl;
n is 0 or 1;
o is 0, 1, 2, 3, or 4;
q is 0, 1, 2, 3, 4, 5, 6, 7, 8, 9, 10, 11, or 12;
x is 0, 1, or 2; and
z is 3, 4, 5, or 6.

11. The compound of claim 10, wherein Y is H.

12. The compound of claim 11, wherein n is 0.

13. The compound of claim 12, wherein R₂ and R₃ are H.

14. The compound of claim 13, and pharmaceutically acceptable salts thereof, wherein the compound is selected from the group consisting of
4-{[4-(3-Methylthien-2-yl)pyrimidin-2-yl]amino}benzenesulfonamide,
3-({4-[4-(Methylsulfonyl)phenyl]pyrimidin-2-yl}amino)benzenesulfonamide,
N-1,3-Thiazol-2-yl-4-[(4-thien-2-ylpyrimidin-2-yl)amino]benzenesulfonamide,
N-Methyl-4-{[4-(1,3-thiazol-2-yl)pyrimidin-2-yl]amino}benzenesulfonamide,
2-Methyl-N-pyrimidin-2-yl-4-{[4-(1,3-thiazol-2-yl)pyrimidin-2-yl]amino}benzenesulfonamide,
4-{[4-(1-Benzothien-2-yl)pyrimidin-2-yl]amino}-N-methyl-N-1,3-thiazol-2-ylbenzenesulfonamide,
2-Methyl-N-pyrimidin-2-yl-4-[(4-thien-3-ylpyrimidin-2-yl)amino]benzenesulfonamide,
N-Isobutyl-4-[(4-pyridin-4-ylpyrimidin-2-yl)amino]benzenesulfonamide,
2-Methyl-4-[(4-pyridin-4-ylpyrimidin-2-yl)amino]-N-pyrimidin-2-ylbenzenesulfonamide,
N-(4-{[2-(Methoxymethyl)pyrrolidin-1-yl]sulfonyl}phenyl)-4-pyridin-4-ylpyrimidin-2-amine,
2-Methyl-N-pyrimidin-2-yl-4-[(4-thien-2-ylpyrimidin-2-yl)amino]benzenesulfonamide,
N-(1-phenyl-1H-pyrazol-5-yl)-4-[(4-thien-2-ylpyrimidin-2-yl)amino]benzenesulfonamide,
N-methyl-N-1,3-thiazol-2-yl-4-[(4-thien-2-ylpyrimidin-2-yl)amino]benzenesulfonamide,
2-methyl-4-{[4-(1-methyl-1H-pyrrol-2-yl)pyrimidin-2-yl]amino}-N-pyrimidin-2-ylbenzenesulfonamide,
N-methyl-4-{[4-(1-methyl-1H-pyrrol-2-yl)pyrimidin-2-yl]amino}-N-1,3-thiazol-2-ylbenzenesulfonamide,
N-[4-(dimethylamino)phenyl]-4-[(4-pyridin-3-ylpyrimidin-2-yl)amino]benzenesulfonamide,
N-methyl-4-[(4-pyridin-3-ylpyrimidin-2-yl)amino]benzenesulfonamide,
2-methyl-4-[(4-pyridin-3-ylpyrimidin-2-yl)amino]-N-pyrimidin-2-ylbenzenesulfonamide,
N-(1-phenyl-1H-pyrazol-5-yl)-4-[(4-pyridin-3-ylpyrimidin-2-yl)amino]benzenesulfonamide,
N-(4-{[2-(methoxymethyl)pyrrolidin-1-yl]sulfonyl}phenyl)-4-pyridin-3-ylpyrimidin-2-amine,
4-{[4-(5-bromothien-2-yl)pyrimidin-2-yl]amino}-N-methyl-N-1,3-thiazol-2-ylbenzenesulfonamide,
N-methyl-4-{[4-(1-naphthyl)pyrimidin-2-yl]amino}-N-1,3-thiazol-2-ylbenzenesulfonamide,
N-(4-{[2-(methoxymethyl)pyrrolidin-1-yl]sulfonyl}phenyl)-4-(1-naphthyl)pyrimidin-2-amine,
N-methyl-4-{[4-(3-methylthien-2-yl)pyrimidin-2-yl]amino}-N-1,3-thiazol-2-ylbenzenesulfonamide,
2-{[(4-{[4-(3-methylthien-2-yl)pyrimidin-2-yl]amino}phenyl)sulfonyl]amino}-1,3-thiazole-4-carboxylic acid,
4-{[(4-pyridin-3-ylpyrimidin-2-yl)amino]methyl}benzenesulfonamide,
4-{[(4-pyridin-4-ylpyrimidin-2-yl)amino]methyl}benzenesulfonamide,
4-({[4-(2-thienyl)pyrimidin-2-yl]amino}methyl)benzenesulfonamide,
3-{[4-(1,3-thiazol-2-yl)pyrimidin-2-yl]amino}benzenesulfonamide,
4-[[4-(1-benzothien-2-yl)pyrimidin-2-yl] (methyl)amino]benzenesulfonamide,
4-[[4-(1-benzothien-2-yl)pyrimidin-2-yl] (methyl)amino]-N-methylbenzenesulfonamide,
3-{[4-(1-benzothien-2-yl)pyrimidin-2-yl]amino}-N-methylbenzenesulfonamide,
3-{[4-(1-benzothien-2-yl)pyrimidin-2-yl]amino}-N-isobutylbenzenesulfonamide, and
4-(4-(naphthalen-2-yl)pyrimidin-2-ylamino)benzenesulfonamide.

15. A compound of the formula (III): 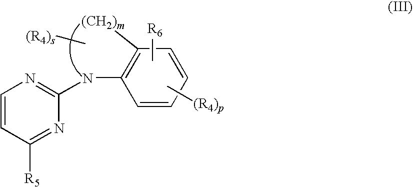
and pharmaceutically acceptable salts thereof,
wherein
R₅ is aryl or heteroaryl both optionally substituted with 1-7 R₄ groups;
R₆ is —SO₂NR₂R₃ or 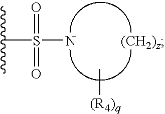
R₂ and R₃ are each independently H, C₁₋₆ alkyl, C₂₋₆ alkenyl, C₂₋₆ alkynyl, C₃₋₆ cycloalkyl, C₃₋₆ cycloalkyl-C₁₋₃ alkyl, aryl, arylalkyl, heteroaryl, or heteroarylalkyl, whereas all except H can be optionally substituted with 1-5 R₄ groups;
each R₄ is independently H, halogen, CN, OH, aryl, arylalkyl, heteroaryl, heteroarylalkyl, C₁₋₆ alkyl, C₂₋₆ alkenyl, C₂₋₆ alkynyl, C₁₋₃ fluorinatedalkyl, C₃₋₆ cycloalkyl, C₃₋₆ cycloalkyl-C₁₋₃ alkyl, NO₂, NH₂, NHC₁₋₆ alkyl, N(C₁₋₆ alkyl)₂, NHC₃₋₆ cycloalkyl, N(C₃₋₆ cycloalkyl)₂, NHC(O)C₁₋₆ alkyl, NHC(O)C₃₋₆ cycloalkyl, NHC(O)NHC₁₋₆ alkyl, NHC(O)NHC₃₋₆ cycloalkyl, SO₂NH₂, SO₂NHC₁₋₆ alkyl, SO₂NHC₃₋₆ cycloalkyl SO₂N(C₁₋₆ alkyl)₂, SO₂N(C₃₋₆ cycloalkyl)₂, NHSO₂C₁₋₆ alkyl, NHSO₂C₃₋₆ cycloalkyl, CO₂C₁₋₆ alkyl, CO₂C₃₋₆ cycloalkyl, CONHC₁₋₆ alkyl, CONHC₃₋₆ cycloalkyl, CON(C₁₋₆ alkyl)₂, CON(C₃₋₆ cycloalkyl)₂OH, OC₁₋₃ alkyl, C₁₋₃ fluorinatedalkyl, OC₃₋₆ cycloalkyl, OC₃₋₆ cycloalkyl-C₁₋₃ alkyl, SH, SOₓC₁₋₃ alkyl, C₃₋₆ cycloalkyl, or SOₓC₃₋₆ cycloalkyl-C₁₋₃ alkyl;
m is 2 or 3;
each p is independently 0, 1, 2, 3, 4, 5, or 6;
q is 0, 1, 2, 3, 4, 5, 6, 7, 8, 9, 10, 11, or 12;
s is 0, 1, 2, 3, 4, 5, or 6;
x is 0, 1, or 2; and
z is 3, 4, 5, or 6.

16. The compound of claim 15, wherein m is 2.

17. The compound of claim 15, wherein m is 3.

18. The compound of claim 15, and pharmaceutically acceptable salts thereof, wherein the compound is selected from the group consisting of
1-(4-(naphthalen-2-yl)pyrimidin-2-yl)-1,2,3,4-tetrahydroquinoline-6-sulfonamide,
1-(4-(thiophen-2-yl)pyrimidin-2-yl)indoline-5-sulfonamide,
1-(4-(naphthalen-2-yl)pyrimidin-2-yl)indoline-5-sulfonamide,
N,N-diethyl-1-(4-(thiophen-2-yl)pyrimidin-2-yl)indoline-5-sulfonamide,
N,N-diethyl-1-(4-(naphthalen-2-yl)pyrimidin-2-yl)indoline-5-sulfonamide,
N,N-dibenzyl-1-(4-(naphthalen-2-yl)pyrimidin-2-yl)indoline-5-sulfonamide,
1-(4-(thiophen-2-yl)pyrimidin-2-yl)-1,2,3,4-tetrahydroquinoline-6-sulfonamide, and
N,N-dimethyl-1-(4-(thiophen-2-yl)pyrimidin-2-yl)indoline-5-sulfonamide.

19. A composition comprising the compound or pharmaceutically acceptable salt of the compound of claim 1 and a pharmaceutically acceptable carrier.

20. The composition of claim 19, wherein the pharmaceutically acceptable carrier is suitable for oral administration and the composition comprises an oral dosage form.

21. A composition comprising the compound or pharmaceutically acceptable salt of the compound of claim 10 and a pharmaceutically acceptable carrier.

22. The composition of claim 21, wherein the pharmaceutically acceptable carrier is suitable for oral administration and the composition comprises an oral dosage form.

23. A composition comprising the compound or pharmaceutically acceptable salt of the compound of claim 15 and a pharmaceutically acceptable carrier.

24. The composition of claim 23, wherein the pharmaceutically acceptable carrier is suitable for oral administration and the composition comprises an oral dosage form.

25. A method of treating a canonical Wnt-β-catenin cellular messaging system related disorder, comprising administering to a mammal in need thereof a compound or a pharmaceutically acceptable salt of a compound of claim 1, 10, or 15 in an amount effective to treat a canonical Wnt-β-catenin cellular messaging system related disorder.

26. The method of claim 25, wherein the canonical Wnt-β-catenin cellular messaging system related disorder is selected from the group consisting of bone disorders, cancer, and Alzheimer's disease.

27. The method of claim 25, wherein the canonical Wnt-β-catenin cellular messaging system related disorder is cancer.

28. The method of claim 27, wherein the cancer is selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer.

29. The method of claim 25, wherein the canonical Wnt-β-catenin cellular messaging system related disorder is Alzheimer's disease.

30. The method of claim 25, wherein the canonical Wnt-β-catenin cellular messaging system related disorder is a bone disorder.

31. The method of claim 30, wherein the bone disorder is selected from the group consisting of osteoarthritis, osteolysis from multiple myeloma, osteoporosis, and rheumatoid arthritis.

32. A method of synthesizing a compound of Formula II, comprising:
reacting a compound of the Formula (IV): 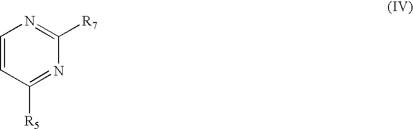
wherein
R₅ is aryl or heteroaryl both optionally substituted with 1-7 R₄ groups; and
R₇ is halogen;
with a compound of Formula (V): 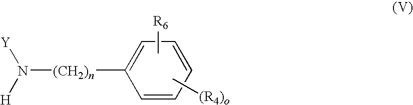
wherein
Y is H, C₁₋₆ alkyl, aryl, or arylalkyl;
R₆ is —SO₂NR₂R₃ or 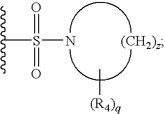
R₂ and R₃ are each independently H, C₁₋₆ alkyl, C₂₋₆ alkenyl, C₂₋₆ alkynyl, C₃₋₆ cycloalkyl, C₃₋₆ cycloalkyl-C₁₋₃ alkyl, aryl, arylalkyl, heteroaryl, or heteroarylalkyl, whereas all except H can be optionally substituted with 1-5 R₅ groups;
each R₄ is independently H, halogen, CN, OH, aryl, arylalkyl, heteroaryl, heteroarylalkyl, C₁₋₆ alkyl, C₂₋₆ alkenyl, C₂₋₆ alkynyl, C₁₋₃ fluorinatedalkyl, C₃₋₆ cycloalkyl, C₃₋₆ cycloalkyl-C₁₋₃ alkyl, NO₂, NH₂, NHC₁₋₆ alkyl, N(C₁₋₆ alkyl)₂, NHC₃₋₆ cycloalkyl, N(C₃₋₆ cycloalkyl)₂, NHC(O)C₁₋₆ alkyl, NHC(O)C₃₋₆ cycloalkyl, NHC(O)NHC₁₋₆ alkyl, NHC(O)NHC₃₋₆ cycloalkyl, SO₂NH₂, SO₂NHC₁₋₆ alkyl, SO₂NHC₃₋₆ cycloalkyl SO₂N(C₁₋₆ alkyl)₂, SO₂N(C₃₋₆ cycloalkyl)₂, NHSO₂C₁₋₆ alkyl, NHSO₂C₃₋₆ cycloalkyl, CO₂C₁₋₆ alkyl, CO₂C₃₋₆ cycloalkyl, CONHC₁₋₆ alkyl, CONHC₃₋₆ cycloalkyl, CON(C₁₋₆ alkyl)₂, CON(C₃₋₆ cycloalkyl)₂OH, OC₁₋₃ alkyl, C₁₋₃ fluorinatedalkyl, OC₃₋₆ cycloalkyl, OC₃₋₆ cycloalkyl-C₁₋₃ alkyl, SH, SOₓC₁₋₃ alkyl, C₃₋₆ cycloalkyl, or SOₓC₃₋₆ cycloalkyl-C₁₋₃ alkyl;
n is 0 or 1;
o is 0, 1, 2, 3, or 4;
q is 0, 1, 2, 3, 4, 5, 6, 7, 8, 9, 10, 11, or 12;
x is 0, 1, or 2; and
z is 3, 4, 5, or 6;
under conditions effective to substitute R₇ with the compound of Formula (V) thereby providing a compound having the Formula (II): 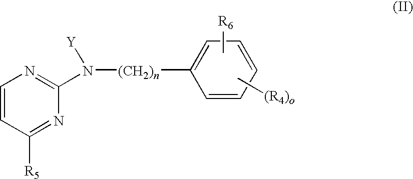
and pharmaceutically acceptable salts thereof,
wherein
R₅ is aryl or heteroaryl both optionally substituted with 1-7 R₄ groups;
Y is H, C₁₋₆ alkyl, aryl, or arylalkyl;
R₆ is —SO₂NR₂R₃ or 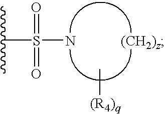
R₂ and R₃ are each independently H, C₁₋₆ alkyl, C₂₋₆ alkenyl, C₂₋₆ alkynyl, C₃₋₆ cycloalkyl, C₃₋₆ cycloalkyl-C₁₋₃ alkyl, aryl, arylalkyl, heteroaryl, or heteroarylalkyl, whereas all except H can be optionally substituted with 1-5 R₄ groups;
each R₄ is independently H, halogen, CN, OH, aryl, arylalkyl, heteroaryl, heteroarylalkyl, C₁₋₆ alkyl, C₂₋₆ alkenyl, C₂₋₆ alkynyl, C₁₋₃ fluorinatedalkyl, C₃₋₆ cycloalkyl, C₃₋₆ cycloalkyl-C₁₋₃ alkyl, NO₂, NH₂, NHC₁₋₆ alkyl, N(C₁₋₆ alkyl)₂, NHC₃₋₆ cycloalkyl, N(C₃₋₆ cycloalkyl)₂, NHC(O)C₁₋₆ alkyl, NHC(O)C₃₋₆ cycloalkyl, NHC(O)NHC₁₋₆ alkyl, NHC(O)NHC₃₋₆ cycloalkyl, SO₂NH₂, SO₂NHC₁₋₆ alkyl, SO₂NHC₃₋₆ cycloalkyl SO₂N(C₁₋₆ alkyl)₂, SO₂N(C₃₋₆ cycloalkyl)₂, NHSO₂C₁₋₆ alkyl, NHSO₂C₃₋₆ cycloalkyl, CO₂C₁₋₆ alkyl, CO₂C₃₋₆ cycloalkyl, CONHC₁₋₆ alkyl, CONHC₃₋₆ cycloalkyl, CON(C₁₋₆ alkyl)₂, CON(C₃₋₆ cycloalkyl)₂OH, OC₁₋₃ alkyl, C₁₋₃ fluorinatedalkyl, OC₃₋₆ cycloalkyl, OC₃₋₆ cycloalkyl-C₁₋₃ alkyl, SH, SOₓC₁₋₃ alkyl, C₃₋₆ cycloalkyl, or SOₓC₃₋₆ cycloalkyl-C₁₋₃ alkyl;
n is 0 or 1;
o is 0, 1, 2, 3, or 4;
q is 0, 1, 2, 3, 4, 5, 6, 7, 8, 9, 10, 11, or 12;
x is 0, 1, or 2; and
z is 3, 4, 5, or 6.

46. A method of synthesizing a compound of Formula (III), comprising: reacting a compound of the Formula (IV): 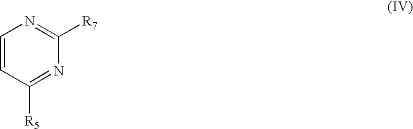
wherein
R₅ is aryl or heteroaryl both optionally substituted with 1-7 R₄ groups; and
R₇ is halogen;
with a compound of Formula (VI): 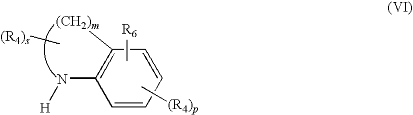
wherein
R₆ is —SO₂NR₂R₃ or 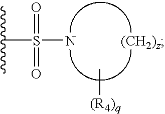
R₂ and R₃ are each independently H, C₁₋₆ alkyl, C₂₋₆ alkenyl, C₂₋₆ alkynyl, C₃₋₆ cycloalkyl, C₃₋₆ cycloalkyl-C₁₋₃ alkyl, aryl, arylalkyl, heteroaryl, or heteroarylalkyl, where as all except H can be optionally substituted with 1-5 R₄ groups;
each R₄ is independently H, halogen, CN, OH, aryl, arylalkyl, heteroaryl, heteroarylalkyl, C₁₋₆ alkyl, C₂₋₆ alkenyl, C₂₋₆ alkynyl, C₁₋₃ fluorinatedalkyl, C₃₋₆ cycloalkyl, C₃₋₆ cycloalkyl-C₁₋₃ alkyl, NO₂, NH₂, NHC₁₋₆ alkyl, N(C₁₋₆ alkyl)₂, NHC₃₋₆ cycloalkyl, N(C₃₋₆ cycloalkyl)₂, NHC(O)C₁₋₆ alkyl, NHC(O)C₃₋₆ cycloalkyl, NHC(O)NHC₁₋₆ alkyl, NHC(O)NHC₃₋₆ cycloalkyl, SO₂NH₂, SO₂NHC₁₋₆ alkyl, SO₂NHC₃₋₆ cycloalkyl SO₂N(C₁₋₆ alkyl)₂, SO₂N(C₃₋₆ cycloalkyl)₂, NHSO₂C₁₋₆ alkyl, NHSO₂C₃₋₆ cycloalkyl, CO₂C₁₋₆ alkyl, CO₂C₃₋₆ cycloalkyl, CONHC₁₋₆ alkyl, CONHC₃₋₆ cycloalkyl, CON(C₁₋₆ alkyl)₂, CON(C₃₋₆ cycloalkyl)₂OH, OC₁₋₃ alkyl, C₁₋₃ fluorinatedalkyl, OC₃₋₆ cycloalkyl, OC₃₋₆ cycloalkyl-C₁₋₃ alkyl, SH, SOₓC₁₋₃ alkyl, C₃₋₆ cycloalkyl, or SOₓC₃₋₆ cycloalkyl-C₁₋₃ alkyl;
m is 2 or 3;
each p is independently 0, 1, 2, 3, 4, 5, or 6;
q is 0, 1, 2, 3, 4, 5, 6, 7, 8, 9, 10, 11, or 12;
s is 0, 1, 2, 3, 4, 5, or 6;
x is 0, 1, or 2; and
z is 3, 4, 5, or 6;
under conditions effective to substitute R₇ with the compound of Formula (VI) thereby providing a compound having the Formula (III): 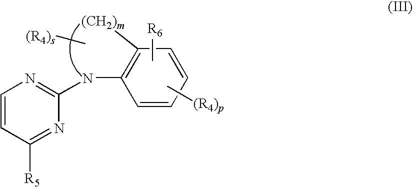
and pharmaceutically acceptable salts thereof,
wherein
R₅ is aryl or heteroaryl both optionally substituted with 1-7 R₄ groups;
R₆ is —SO₂NR₂R₃ or 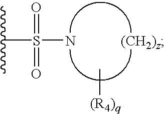
R₂ and R₃ are each independently H, C₁₋₆ alkyl, C₂₋₆ alkenyl, C₂₋₆ alkynyl, C₃₋₆ cycloalkyl, C₃₋₆ cycloalkyl-C₁₋₃ alkyl, aryl, arylalkyl, heteroaryl, or heteroarylalkyl, whereas all except H can be optionally substituted with 1-5 R₄ groups;
each R₄ is independently H, halogen, CN, OH, aryl, arylalkyl, heteroaryl, heteroarylalkyl, C₁₋₆ alkyl, C₂₋₆ alkenyl, C₂₋₆ alkynyl, C₁₋₃ fluorinatedalkyl, C₃₋₆ cycloalkyl, C₃₋₆ cycloalkyl-C₁₋₃ alkyl, NO₂, NH₂, NHC₁₋₆ alkyl, N(C₁₋₆ alkyl)₂, NHC₃₋₆ cycloalkyl, N(C₃₋₆ cycloalkyl)₂, NHC(O)C₁₋₆ alkyl, NHC(O)C₃₋₆ cycloalkyl, NHC(O)NHC₁₋₆ alkyl, NHC(O)NHC₃₋₆ cycloalkyl, SO₂NH₂, SO₂NHC₁₋₆ alkyl, SO₂NHC₃₋₆ cycloalkyl SO₂N(C₁₋₆ alkyl)₂, SO₂N(C₃₋₆ cycloalkyl)₂, NHSO₂C₁₋₆ alkyl, NHSO₂C₃₋₆ cycloalkyl, CO₂C₁₋₆ alkyl, CO₂C₃₋₆ cycloalkyl, CONHC₁₋₆ alkyl, CONHC₃₋₆ cycloalkyl, CON(C₁₋₆ alkyl)₂, CON(C₃₋₆ cycloalkyl)₂OH, OC₁₋₃ alkyl, C₁₋₃ fluorinatedalkyl, OC₃₋₆ cycloalkyl, OC₃₋₆ cycloalkyl-C₁₋₃ alkyl, SH, SOₓC₁₋₃ alkyl, C₃₋₆ cycloalkyl, or SOₓC₃₋₆ cycloalkyl-C₁₋₃ alkyl;
m is 2 or 3;
each p is independently 0, 1, 2, 3, 4, 5, or 6;
q is 0, 1, 2, 3, 4, 5, 6, 7, 8, 9, 10, 11, or 12;
s is 0, 1, 2, 3, 4, 5, or 6;
x is 0, 1, or 2; and
z is 3, 4, 5, or 6.
